(19) United States
(12) Patent Application Publication
(10) Pub. No.: US 2026/0199555 A1
(43) Pub. Date: Jul. 16, 2026

(54) 3D PRINTING OF HIGH CELL DENSITY VASCULARIZED TISSUE

(71) Applicant: THE REGENTS OF THE UNIVERSITY OF CALIFORNIA, Oakland, CA (US)

(72) Inventors: Shangting YOU, La Jolla, CA (US); Shaochen CHEN, San Diego, CA (US); Yi XIANG, La Jolla, CA (US)

(21) Appl. No.: 19/133,673

(22) PCT Filed: Nov. 30, 2023
(86) PCT No.: PCT/US2023/081860

§ 371 Date: May 28, 2025

Related U.S. Application Data
(60) Provisional application No. 63/428,981, filed on Nov. 30, 2022.

(51) Int. Cl. A61L 27/38; A61L 27/22; A61L 27/50; B29C 64/314; B33Y 10/00; B33Y 70/00; C08L 89/06

(52) U.S. Cl. CPC A61L 27/38; A61L 27/222; A61L 27/50; B29C 64/314; B33Y 10/00; B33Y 70/00; C08L 89/06

(57) ABSTRACT

A composition and method for 3D bioprinting adds a contrast agent having a relatively high refractive index to a photopolymerizable bioink composition in which cells are encapsulated so that the refractive index of the composition substantially matches the refractive index of the encapsulated cells.

### RELATED APPLICATIONS

This application claims the benefit of the priority of U.S. Provisional Application No. 63/428,981, filed Nov. 30, 2022, which is incorporated herein by reference in its entirety.

### GOVERNMENT RIGHTS

This invention was made with government support under Grants CA253615, HD100132, and EB021857 awarded by the National Institutes of Health and Grants 2135720 and 1903933 awarded by the National Science Foundation. The government has certain rights in the invention.

### BACKGROUND

Large-scale 3D engineered tissues with high cell density and fine resolution that accurately resemble native biological tissues and organs are highly sought after in biomedical research and clinical applications. 3D bioprinting techniques have emerged as the most popular methods to fabricate artificial tissues, however, there are challenges in simultaneously satisfying the requirements of high cell density, high cell viability, and fine fabrication resolution. In one example, bioprinting resolution of digital light processing (DLP)-based 3D bioprinting suffers with increasing bioink cell density because the large number of cells encapsulated in the bioink severely scatters incident light.

3D engineered tissues are artificial functional bionic tissues comprised of biomaterial scaffolds and living cells. Engineered tissues have found many biomedical applications including basic biomedical research, disease modeling, drug testing, personalized medicine, regenerative medicine, and organ transplantation. 3D engineered tissues can accurately recapitulate the 3D architecture, cell types, physical and biochemical environment of the native tissues, providing in vitro tissue or organ models with better bio-relevancy, scalability, and reproducibility compared to traditional 2D monolayer cell models or animal models. Furthermore, 3D engineered transplantable tissues and organs developed with autologous cells can potentially mitigate the problems associated with organ donor shortage and immune rejection. Therefore, tissue engineering has attracted significant research interest.

Native human tissues typically have a cell density on the order of 1-3 billion/mL and include complex 3D structures with micron-scale features. To closely recapitulate the native tissues, high cell density (HCD) is essential in many 3D engineered tissues to build cell-cell interactions, which are critical for the artificial tissue to function. For instance, 3D engineered cardiac tissue typically requires a cell density greater than 40 million/mL to enable spontaneous contraction of the tissue. Additionally, HCD ensures physiological compatibility, potentially allowing functional artificial organs for implantation. Currently, the typical cell density used in tissue engineering research is around 1 to 10 million/ml, which is two or three orders of magnitude lower than native tissues.

Apart from cell density, fine microscale features are also critical to the native tissues' viability and proper function. For instance, vasculature networks are essential in tissues and organs for nutrient and gas exchange. The diffusion limit for nutrient and gas exchange is between 200 to 300 microns. Traditionally, the inability to fabricate perfusable vasculature networks in conjunction with the tissues of interest limits the thickness of engineered tissues by this diffusion limit. However, high-resolution 3D bioprinting enables fabrication of vasculature networks within 3D engineered tissues to support the cell viability in the thick tissues. Hence, any improvement of biofabrication resolution can lead to transitioning away from thin tissue engineered constructs to large-scale 3D engineered tissues and even transplantable organs. Accordingly, there is a need to simultaneously achieve high cell density and high resolution in 3D engineered tissues.

3D bioprinting has emerged as the most popular method to fabricate 3D engineered tissues, due to its ability to precisely deposit multiple cells and biomaterials in user-defined shapes. Various 3D bioprinting techniques have been developed in recent years, which can be broadly classified into two categories: extrusion-based printing and light-based printing. See, e.g., W. Zhu, et al., “3D printing of functional biomaterials for tissue engineering”, Current Opinion in Biotechnology, 40, 103-112 (2016), incorporated herein by reference. Extrusion-based bioprinting methods, including nozzle-extrusion and ink-jet methods, selectively deposit a bioink to the desired location to build a 3D construct. Due to the limitation of the physical size of the nozzle or ink-jet head, the best fabrication resolution that can be achieved is typically on the order of ~50 μm. By contrast, light-based bioprinting methods, including stereolithography, two-photon polymerization (2PP), and digital light processing (DLP) methods, selectively deliver photon energy to the desired location to locally crosslink (solidify) a bioink to fabricate a 3D structure. Since light can be precisely manipulated by optical lenses and are not limited by physical apertures, light-based bioprinting methods can achieve micron scale or even sub-micron scale nominal resolutions.

Although a 50 μm nominal resolution can be achieved by extrusion-based printing, and micron-scale nominal resolution can be achieved by light-based printing, such fine features are often achievable only under the specific conditions optimized for fabrication, where low-biocompatibility materials without encapsulated cells are used. In actual bioprinting applications where cell-encapsulated bioinks are used, the fabrication resolution substantially deteriorates compared to the nominal situation. For extrusion-based 3D bioprinting, increasing cell density or using spheroids in the bioink requires use of a larger nozzle tip to avoid impacting cell viability due to the shear stresses during extrusion. Typically, for 10 million/mL or higher cell density, a 200 μm or larger nozzle tip should be used, where the resulting printing resolution ranges between 200 μm-500 μm. For light-based methods, the light scattering caused by the cells degrades resolution, where typical resolution is a few tens of microns to a few hundreds of microns. While some chemical additives such as TEMPO can mitigate the unwanted polymerization caused by light scattering, these chemicals tend to be cytotoxic. Thus, it is difficult to fabricate a 3D bioprinted structure that simultaneously has high cell density (≥20 million/mL), high cell viability (≥80%), and high fabrication resolution (≤50 μm). This problem can be referred to as the “Density-Viability-Resolution Trilemma” in 3D bioprinting.

HCD promotes physiological compatibility, potentially enabling the implantation of functional bioartificial organs. For instance, 3D-engineered cardiac tissue typically requires a cell density greater than 40 million/ml to enable spontaneous contraction of the tissue. The cell density employed in tissue engineering research today is typically 1-20 million/ml, which is much less than native tissues. Fine microscale topological features, in addition to cell density, are essential for the viability and proper function of native tissues. For example, the exchange of nutrients and gases in tissues and organs heavily depends on the networks of fine vasculatures. The diffusion limit for nutrient and gas exchange is between 200 to 300 μm. Traditionally, the thickness of engineered tissues has been constrained by this diffusion limit since it is difficult to fabricate perfusable vascular networks together with the tissues of interest. Therefore, developing novel methods that could simultaneously achieve HCD and high resolution while maintaining good cell viability in 3D-engineered tissues will have a significant impact on tissue engineering and regenerative medicine.

In the area of liver diseases, approximately 40,000 people in the U.S. die each year from acute or chronic liver diseases. Organ transplantation is the gold standard of care for end-stage liver diseases. Unfortunately, the dramatic discrepancy between available donors vs. patients on the waiting lists highlights the critical need for functional liver replacements. Furthermore, as the liver serves a vital role in drug metabolism and detoxification, the investigation of liver-drug interactions is an essential component of any preclinical drug study. Conventional animal models are costly, often unreliable, and difficult to translate to human studies due to the species-specific variations in hepatocellular functions. For example, in 2004, the FDA estimated that 92 out of every 100 drugs that successfully passed preclinical animal testing failed in subsequent human trials. As a result, the financial consequence of a drug failing at the clinical stage is often catastrophic to the drug makers. Several human liver models, such as liver slices, microsomes, and cell line and primary hepatocytes are currently in use, however, these models are still limited in their ability to fully represent the dynamic cellular responses of a functional liver tissue.

Recently, liver tissue engineering has significantly progressed toward creating in vitro 3D liver models for drug screening. Human iPSCs may offer an unlimited supply of hepatocytes from various donors, enhancing experimental reproducibility and enabling the study of individualized hepatotoxicity. However, with improvements in cell sourcing, the development of fully functional liver constructs has been limited due to the challenge in fully recapitulating the native physical structure of a hepatic tissue. Such micro-architecture plays a critical role in stem cell biology and hepatocellular function. U.S. Pat. No. 10,954,489 of Qu, et al., incorporated herein by reference, describes the use of DMD-based bioprinting to fabricate a liver-mimetic structure that was effective in neutralization of toxins. Nonetheless, actual replication of tissue morphologies involves the consideration of both their complex 3D geometries, the heterogeneity of their constituent cell populations, as well as appropriate cell densities to ensure cell-cell interactions.

The mechanical properties of the extracellular matrix (ECM) are known to be crucial in influencing various biological processes, impacting cell phenotype and behavior. Hydrogels have been employed effectively as ECM mimics due to their biocompatibility and a tunable range of stiffness that resembles real tissue. Utilizing digital light processing (DLP) printing, photocrosslinkable hydrogels can be fabricated into complex 3D structures. The curing of these hydrogels occurs through free-radical polymerization, where factors such as light power, exposure time, and the concentrations of both the precursor solution and the photoinitiator significantly affect the stiffness of the resulting material.

Photocrosslinkable biopolymers derived from natural materials, such as methacrylated gelatin (GelMA) from porcine skin, show better biocompatibility and biodegradability with mammalian cells compared to synthetic polymers, enabling wide application in tissue engineering. However, these biopolymers, being synthesized by biological organisms, face challenges such as significant batch-to-batch variation. This variability presents obstacles in scaling up research findings for broader applications.

### SUMMARY

The inventive approach mitigates scattering-induced deterioration of bioprinting resolution by incorporating an additive that modifies the refractive index of the bioink to better match the refractive index of any encapsulated cells. Fine-tuning of the refractive index enables a 10-fold reduction in light scattering and a significant increase in fabrication resolution of high-cell-density bioinks. Generally, the additive is a biocompatible water-soluble contrast agent. In some embodiments, the additive may be iodixanol (IDX), a nonionic hydrophilic iodine-containing compound commonly used as a radiocontrast agent during coronary angiography. The inventive approach is highly biocompatible-test results indicated no statistically significant effect on cell viability or phenotype. 50 μm fabrication resolution was achieved in a bioink with 0.1 billion/mL cell density. To evaluate application to tissue/organ 3D bioprinting, large-scale, high-cell-density tissues with fine vascular networks were fabricated. The tissues were viable in a perfusion culture system, with endothelialization and angiogenesis observed after 14 days of culture.

The inventive approach enables high cell density, high cell viability, and fine resolution simultaneously, thus representing a major step towards the fabrication of functional large-scale clinically transplantable tissues or organs, where high cell density and fine vascular networks are essential. Applications of the inventive approach go beyond mammalian and non-mammalian tissue/organ fabrication, enabling the bioprinting of plant, algal, fungal, bacterial, and archaea cells and tissue.

DLP-based 3D bioprinting has emerged as a promising biofabrication technique due to its high resolution, high cell viability, and rapid speed. The inventive approach provides a solution to the Density-Viability-Resolution Trilemma in DLP-based 3D bioprinting. By incorporating an additive with a higher refractive index than that of the bioink itself, it is possible to precisely tune its refractive index to match that of the encapsulated cells' cytoplasm. In exemplary embodiments, iodixanol (IDX) may be used, however, other known contrast agents may be used. With refractive index modification, scattering caused by the mismatch of refractive index between the cells and their surrounding biomaterials can be minimized. Measurement of the bioinks' optical properties and simulation of light propagation confirm that IDX can effectively tune the refractive index of the bioink, and thus significantly reduce light scattering caused by the encapsulated cells by ~10 fold. Testing with IDX enabled bioprinting with a very high cell density (0.1 billion/mL) with a fabrication resolution of 50 microns. Immunofluorescence images and RNA sequencing also confirm that healthy and functional 3D engineered tissues can be fabricated using this approach. No statistically significant change in cells' viability, proliferation, or phenotype was observed when incorporating IDX in the bioink. Furthermore, thick pre-vascularized tissues, with an overall size of 17×11×3.6 mm and vascular channel diameters ranging from 250 μm to 600 μm, and with 40 million/mL cell density, can be fabricated. Endothelialization and angiogenesis were observed in these tissues after 14 days of perfusion culture.

The addition of a contrast agent to the bioink to tune the refractive index to more closely match that of the cells' cytoplasm provides a significant improvement in resolution, however, this approach cannot completely eliminate differences in the refractive indices. The concentration of the contrast agent must be adjusted based on cell type, as different cells exhibit variance in refractive index due to their organelle composition. Consequently, the hydrogel precursor composition is also varied to match each cell type's optimal contrast agent concentration. All these conditions will lead to the variation of the mechanical properties such as stiffness of the bioprinted construct. Although it is possible to measure the stiffness for each cell type under comprehensive printing conditions, a significant challenge arises when dealing with precious cells, such as primary cells isolated from tissues. These cells are often available in limited quantities, insufficient for detailed stiffness measurements under various printing conditions. Therefore, there is a pressing need to predict a wide range of stiffnesses based on printing parameters, or to determine the necessary printing parameters for achieving specific stiffnesses, by utilizing only a limited number of data points and a minimal amount of these valuable cells.

To enable prediction of construct stiffness in HCD printing based on cell density and light exposure time, a neural network (NN) machine learning model can be employed. Initially, stiffness results under detailed printing conditions are obtained using a low-cost 293T cell line. After training the model with data from 293T cells, transfer learning can be implemented to predict the stiffness of a model cell, HepG2 cells, using only a few data points from HepG2 under limited printing conditions. This model demonstrates a good fit with the stiffnesses from 293T cells and effectively predicts the stiffness of HepG2 cells with limited input data.

In one aspect, a method for improved resolution of 3D bioprinted tissue includes: adding to a photopolymerizable bioink composition having cells encapsulated therein a contrast agent, wherein the bioink composition has a first refractive index and the contrast agent has a second, higher refractive and is added in an amount sufficient to cause the composition to substantially match a refractive index of the encapsulated cells. In some embodiments, the contrast agent is biocompatible water-soluble contrast agent. The contrast agent may be selected from iodixanol, iohexol, iopamidol, iopromide, ioversol, iopromide and ioversol. The contrast agent may have a concentration within a range of about 1 to 50 percent. In embodiments where the contrast agent is iodixanol, concentration may be within a range of 20 to 35 percent.

The encapsulated cells may be selected from cell types consisting of mammalian, non-mammalian, plant, algae, fungal, bacterial, and archaea. and may have a cell density in a range of 5 million/mL to 5 billion/mL. The cell density may be configured to produce a selected tissue stiffness.

The bioink composition may be selected from methacrylated gelatin (GelMA), glycidyl methacrylate hyaluronic acid (GMHA), alginate methacrylate (AlgMA), polyethylene glycol (PEG), gelatin-norbornene (Gel-NB), Gelatin-thiol (Gel-SH), and combinations thereof. In some embodiments, the bioink composition is 0.05% to 100% (w/V) GelMA in a solvent.

In another aspect, a composition for 3D bioprinting of tissue includes a mixture of: a photopolymerizable bioink having a first refractive index; cells comprising cytoplasm having a second refractive index higher than the first refractive index; and a contrast agent having a third refractive index higher than the first refractive index, wherein the contrast agent is added in a sufficient amount to substantially match a refractive index of the composition to the second refractive index. In some embodiments, the contrast agent is biocompatible water-soluble contrast agent. The contrast agent may be selected from iodixanol, iohexol, iopamidol, iopromide, ioversol, iopromide and ioversol. The contrast agent may have a concentration within a range of about 1 to 100% (w/V). In embodiments where the contrast agent is iodixanol, concentration may be within a range of 20 to 35 percent.

The encapsulated cells may be selected from cell types consisting of mammalian, non-mammalian, plant, algae, fungal, bacterial, and archaea. and may have a cell density in a range of 5 million/mL to 5 billion/mL. The cell density may be configured to produce a selected tissue stiffness.

The bioink composition may be selected from methacrylated gelatin (GelMA), glycidyl methacrylate hyaluronic acid (GMHA), alginate methacrylate (AlgMA), polyethylene glycol (PEG), gelatin-norbornene (Gel-NB), Gelatin-thiol (Gel-SH), and combinations thereof. In some embodiments, the bioink composition is 0.05% to 50% (w/V) GelMA in a solvent.

Refractive-index-matching-bioink with a contrast agent allows a significantly increased range of cell density tunable (0-200 million cell/mL) in light based bioprinting without compromising the resolution (~50 μm), such as, DLP, 2PP, light sheet, volumetric. For evaluation, the contrast agent iodixanol, or IDX, was used. The expanded range enables cell density as a variable to be controlled in the bioprinting-based tissue engineering to achieve desired tissue organization, parallel to other biochemical/biophysical cues such as stiffness, geometry, incorporation of growth factors, etc.

Specifically, for parenchymal tissue engineering, such as muscle, myocardium, alveoli, hepatic parenchyma, nephrons, bioprinting with a high cell density of the relevant cells substantially improve the tissue reorganization and functional recovery by facilitating sufficient cell-cell interaction.

For non-parenchymal tissue engineering, such as epithelium, mesothelium, bone, cartilage, dermis, bioprinting with an expanded cell density allows for controlled cell proliferation, differentiation, and reorganization, typically through controlled activation of Hippo-YAP (yes-associated protein) signaling. Specifically, cell density can be used to modulate apical extrusion, epithelial-mesenchymal transition for epithelial tissue engineering. Cell density can also be used to guide mesenchymal stem cell differentiation toward osteogenesis, adipogenesis or chondrogenesis in orthopedic tissue engineering. In chondrogenesis engineering, high or low cell density can lead to elastic cartilage or fibrocartilage, respectively. For dermal tissue engineering, cell density can be tuned to facilitate the modeling of fibrosis or healthy regeneration.

The approach disclosed herein employs contrast agent-based refractive-index-matching-bioink to realize high-resolution, varied cell density in light-based bioprinting to achieve improved tissue reorganization for parenchymal tissue engineering, and controlled tissue reorganization for non-parenchymal tissue engineering.

### DESCRIPTION OF THE DRAWINGS

FIG. 1A diagrammatically compares light propagation in a refractive-index-unmatched bioink (upper panel) versus a matched bioink (lower panel); FIG. 1B diagrammatically illustrates a DLP-based 3D bioprinter; FIG. 1C provides a printing resolution comparison among three different bioink compositions: bioink without cells, bioink with 0.1 billion/mL cells, and refractive-index-matched bioink with 0.1 billion/mL cells.

FIGS. 2A-2F illustrate aspects of the optical properties and light energy distribution, where FIG. 2A shows refractive index of 5% GelMA bioink with various IDX concentrations, and the refractive index of OptiPrep™ solution (60% IDX); FIGS. 2B-2D, respectively, show scattering coefficient, anisotropy, and reduced scattering coefficient of 40 million/mL cell-encapsulated bioink with various IDX concentrations; FIG. 2E compares simulated and measured angular distribution for bioink with 0% and 30% IDX; and FIG. 2F provides a comparison of the projected pattern at different depths of the cell-encapsulated bioink with 0% IDX (upper panel) and 30% IDX (lower panel).

FIGS. 3A-3G illustrate biocompatibility analysis, where FIGS. 3A-3C compare metabolic strength of the printed tissues using bioinks with or without IDX; FIG. 3D compares live cell percentage of the printed tissues using bioinks with or without IDX; FIG. 3E provides PCA results of HUVEC slabs using bioinks with or without IDX (n=3); FIG. 3F provides network analysis of enriched gene sets in the MSigDB curated collection; and FIG. 3G shows network analysis of enriched gene sets in the ontology collection, where dark nodes represent upregulated gene sets and white nodes represent downregulated gene sets.

FIGS. 4A-4D illustrate 3D printing of vascularized perfusable thick tissues where FIG. 4A is a schematic of the perfusion culture system and the 3D render of the printed tissue; FIG. 4B provides μCT images of the printed samples (perspective view and cross-sections); FIG. 4C provides bright-field images of the printed samples (top view (left) and cross-section (right)); and FIG. 4D plots cell viability in the thick tissue after 14 days of perfusion culture.

FIG. 5A is a schematic showing the setup of the refractometer for refractive index measurement; FIG. 5B plots the critical angle of total internal refraction based on images collected by the camera in FIG. 5A.

FIG. 6 is a diagrammatic view of a manifold used in the perfusion culture system of FIG. 4A.

FIGS. 7A-7B are plots of refractive indices of common bioinks at various biopolymer and IDX concentrations, respectively.

FIG. 8A plots rheology properties of the bioinks with or without IDX; FIG. 8B plots Young's moduli of the printed structure with 5% GelMA, 30% IDX, and cell density of 0, 40 million/mL, 0.1 billion/mL, and 0.225 billion/mL.

FIG. 9A plots of cell viability at differential hepatocyte density (H, M, L) at various time points (days 0, 1, 4 and 7); FIG. 9B provides fluorescent images of live/dead staining of differential hepatocyte density after one week culture.

FIGS. 10A-10D plot the results of analysis of differential hepatocyte density for two different markers, where FIGS. 10A and 10B plot the % positive hepatocytes for E-cadherin and ZO1, respectively, measured by flow cytometry; FIG. 10C plots albumin secretion levels of hepatocytes in three different conditions over time; and FIG. 10D plots urea secretion levels of hepatocytes in three different conditions over time.

FIGS. 11A-11B provide results of metabolic function analysis, where FIG. 11A is a volcano plot of statistically significant differentially expressed genes at Q<0.05 identified from the RNA-Seq libraries in HCD and MCD; and FIG. 11B plots of gene expression levels of ALB, HNF4a, TTR, AFP, TJP1 and CDH1 for hepatocytes at different densities.

FIG. 12A illustrates the main KEGG pathway analyses between HCD and MCD models based on RNA-seq. Starred descriptions represent liver related pathway; FIG. 12B plots gene expression levels of CYP family (CYP1A2, CYP2B6, CYP2C9 and CYP3A4 for high, medium, and low cell density.

FIG. 13 plots gene expression levels of YAP related gene (YAP1, WWTR1, CTGF and CYR61) in HCD and MCD samples.

FIGS. 14A-14B are plots of refractive index of a 5% GelMA bioink solution with varying concentrations of contrast agents ioversol and iohexol, respectively, as measured using a brix refractometer; FIGS. 14C-14D show the refractive index converted from the Brix % data plotted in FIGS. 14A-14B.

### DETAILED DESCRIPTION OF EMBODIMENTS

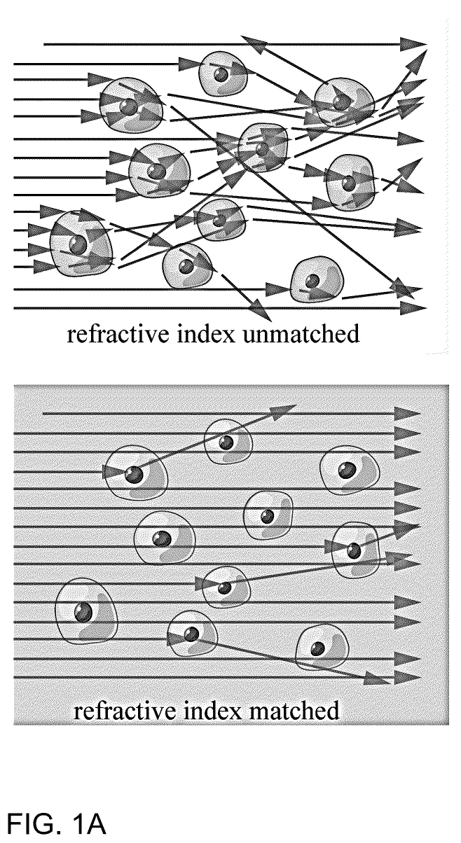

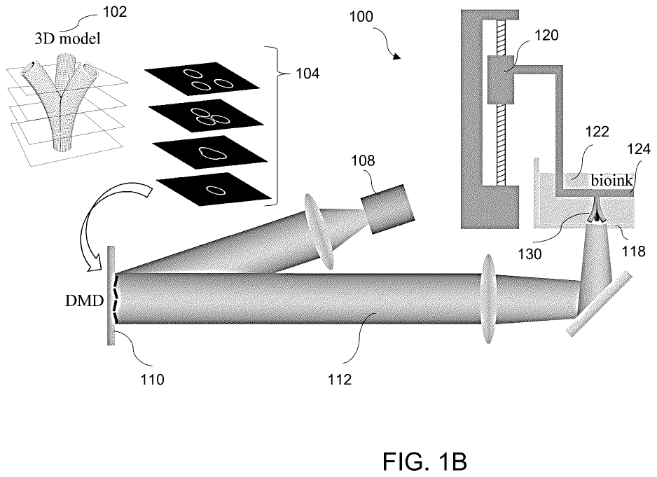

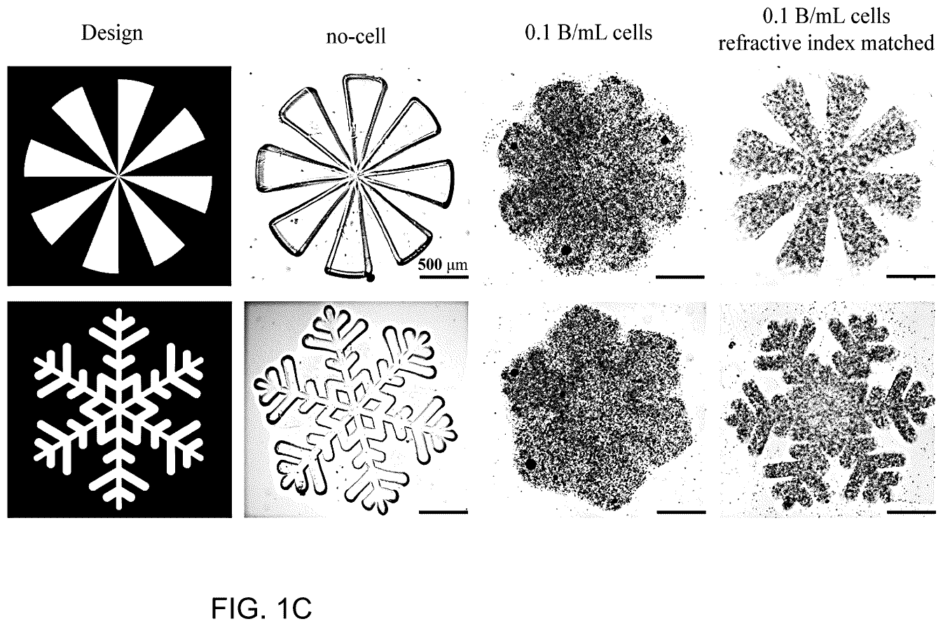

Referring to FIG. 1B, the DLP-based 3D bioprinter 100 projects light from light source 108 onto a digital micromirror device (DMD) 110 which modulates the light using a series of 2D cross-sectional masks 104 formed from slices of a 3D model 102 of the structure to be printed. The modulated light is projected along optical path 112, which includes optical elements (mirrors, lenses, etc.) into the photocrosslinkable bioink 122 contained within container 118, which has a light-transmissive bottom. The modulated light pattern is focused onto the lower surface of stage 124, which is suspended within the bioink. Upon light exposure, the photocrosslinkable bioinks, which can be either synthetic or natural, are solidified. Next, the motorized stage 120 lifts up by one layer thickness (typically a few tens microns to 200 microns) to allow uncured bioink to refill the gap. Subsequently, the next cross-section is projected to the bioink and a new layer is solidified. The optical pattern can be programmed to dynamically change over time as the z-stage lifts up to allow uncured bioink to refill the gap for continuous photo-polymerization, which ultimately results in the fabrication of the designed 3D construct. Due to its scanningless and continuous photo-polymerization process, this bioprinting method can fabricate a sizable tissue construct (i.e., several cm) in a few minutes with a high structural integrity and microscale resolution. In a traditional bioprinting process (e.g., extrusion), the 3D construct is stacked line-by-line or dot-by-dot, resulting in artificial gaps within the construct. These gaps weaken the structural integrity, especially when the cell density is high. The inventive approach solves this problem since the 3D construct is printed in a scanningless fashion within a layer and continuously between layers, completely eliminating the artificial gaps. Further detail of the DLP-based 3D bioprinter can be found in the disclosure of U.S. Pat. No. 10,464,307 of Chung, et al., which is incorporated herein by reference.

By repeating this process, a 3D structure 130 can be fabricated based on the 3D model 102. In ideal conditions, a newly formed layer should exactly match the shape of the projected cross-section (104). However, in practice, the incorporation of cells in the bioink causes severe light scattering, resulting in blurring of the projected light in the bioink. Consequently, the newly formed layers are unable to replicate the fine details of the projected cross-sections.

By tuning the refractive index of the bioink, the scattering effect caused by the cells in the bioink can be minimized, and the fabrication resolution can be significantly improved. This tuning is achieved by adding to the bioink a solution having a higher refractive index than that of the bioink itself. Generally, the additive may be a biocompatible water-soluble contrast agent, such as iodinated contrast agents used to enhance the ability to see blood vessels and organs in radiographic imaging. A few examples of appropriate commercially available, FDA-approved contrast agents include iodixanol (“Visipaque” or “OptiPrep”), iohexol (“Omnipaque”), iopamidol (“Isovue”), iopromide (“Ultravist”), and ioversol (“Optiray”), Iopromide (“Ultravist”) and ioversol (“Optiray”). Selection of other possible contrast agents will be apparent to those of skill in the art based on the disclosure herein. For evaluation of the inventive method, the additive used was iodixanol, referred to herein as “IDX”, a nonionic hydrophilic iodine-containing compound commonly used as a radiocontrast agent during coronary angiography. Testing found that the inventive scheme was able to achieve ~50 μm feature size in a refractive-index-matched gelatin methacrylate (GelMA) bioink with a cell density as high as 0.1 billion/mL. In some embodiments, the bioink may be 0.1% to 15% (w/V) GelMA in a solvent, which is typically PBS.

To evaluate resolution, spoke-shaped and snowflake-shaped 3D structures of 250 um thickness were designed, and 3D printed with the DLP-based 3D bioprinter using varying bioink compositions: (a) without cells, (b) with 0.1 billion/mL cells, and (c) refractive-index-matched bioink with 0.1 billion/mL cells, respectively. The acellular bioink (a) consists of 5% (w/V) GelMA, 0.6% lithium phenyl-2,4,6-trimethylbenzoylphosphinate (LAP), and phosphate-buffered saline (PBS) as solvent. The cell-laden bioink (b) consists of 5% GelMA, 0.6% LAP, 0.1 billion/mL 293T cells, and PBS as solvent. The cell-laden refractive-index-matched bioink (c) consists of 5% GelMA, 0.6% LAP, 0.1 billion/mL 293T cells, 30% IDX, and PBS as solvent.

FIG. 1C shows the bright field microscopic images of the printing results, with the spoke pattern in the upper row and the snowflake pattern in the lower row. The first column provides the original design. The acellular bioink (a) (2^{nd} column) exhibits the best printing resolution while the bioink with 0.1 billion/mL cells (3^{rd} column) cannot resolve either the spoke or snowflake shape due to light scattering. However, by tuning the refractive index of this high-cell-density bioink, the resolution can be significantly improved, and many of the fine details of the designed structure can be resolved, as seen in the 4^{th} column. Both positive and negative features (void spaces) of ~50 μm size can be resolved in this manner. These results demonstrate that tuning the refractive index of the bioinks can effectively improve the fabrication resolution, especially in high-cell-density bioinks. Nonetheless, the achieved resolution by matching the index of refraction can likely be further optimized depending on the cell density, material composition, structural complexity, and other key features unique to the tissue of interest.

Light scattering in cell-laden bioinks can originate from Rayleigh scattering and Mie scattering. Natural or modified macromolecules such as hyaluronic acid, gelatin, and collagen are commonly used in bioinks, which give rise to Rayleigh scattering. Subcellular components such as nucleus and organelles cause Mie scattering. Most importantly, the cytoplasm typically has a higher refractive index than the bioink, resulting in the situation where each cell can deflect photons passing through it, similar to a microscopic lens, causing severe Mie scattering.

Cytoplasm typically has a refractive index between 1.36 and 1.39, whereas hydrogel bioinks typically have a refractive index close to that of water (1.33). IDX solution has a refractive index of greater than 1.4. Thus, by adding IDX to the bioink, the refractive index of the bioink can be tuned to match that of the cytoplasm. Therefore, scattering caused by the mismatch of refractive index between the cytoplasm and environment can be minimized, as illustrated diagrammatically in FIG. 1A.

The concentration of the contrast agent in the bioink solution may range from about 1 to 100% w/V. Identifying an optimal concentration of IDX in the final bioink solution depends on many factors, including the bioink composition, encapsulated cells' type(s), osmolarity, temperature, light wavelength, etc. A pilot experiment is recommended to optimize the concentration of contrast agent for a given application. Since the cytoplasm typically has a refractive index between 1.36 and 1.39, the optimal IDX concentration may from 20%~35% w/V.

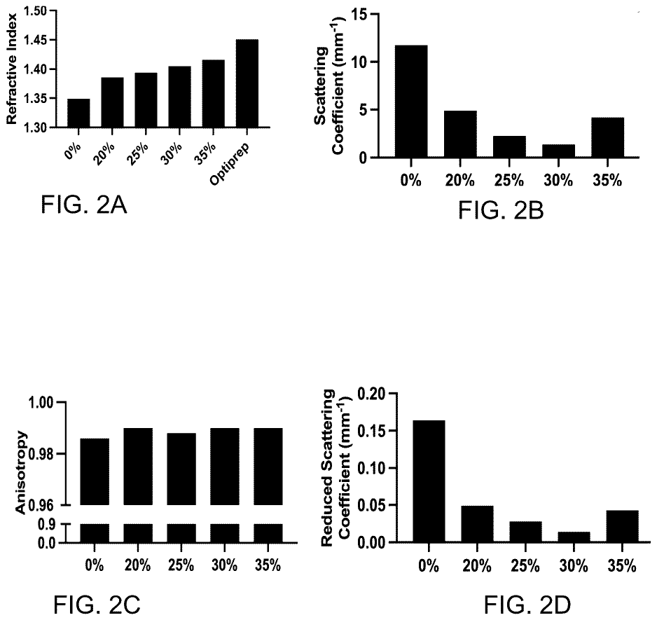

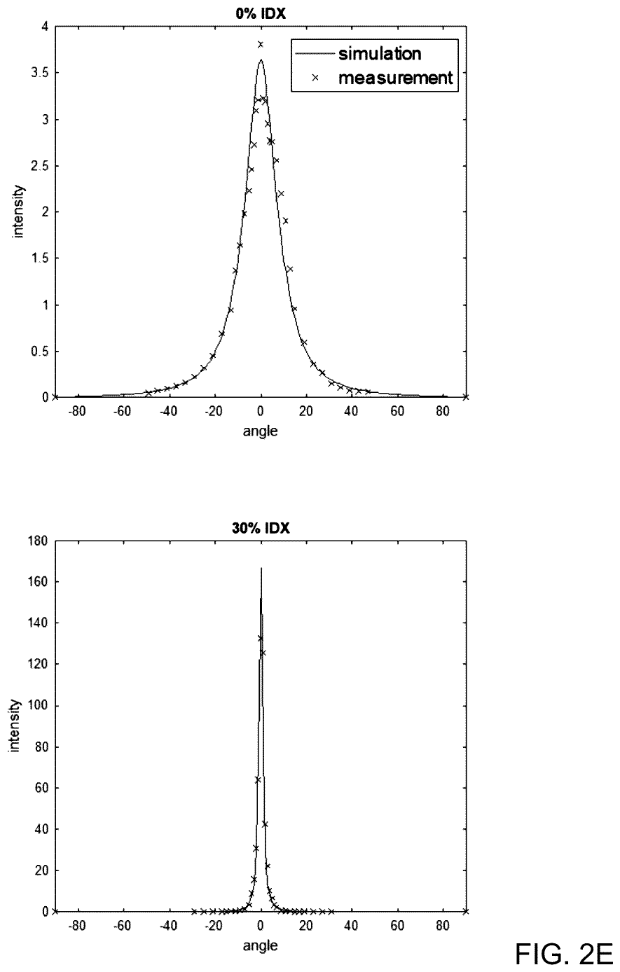

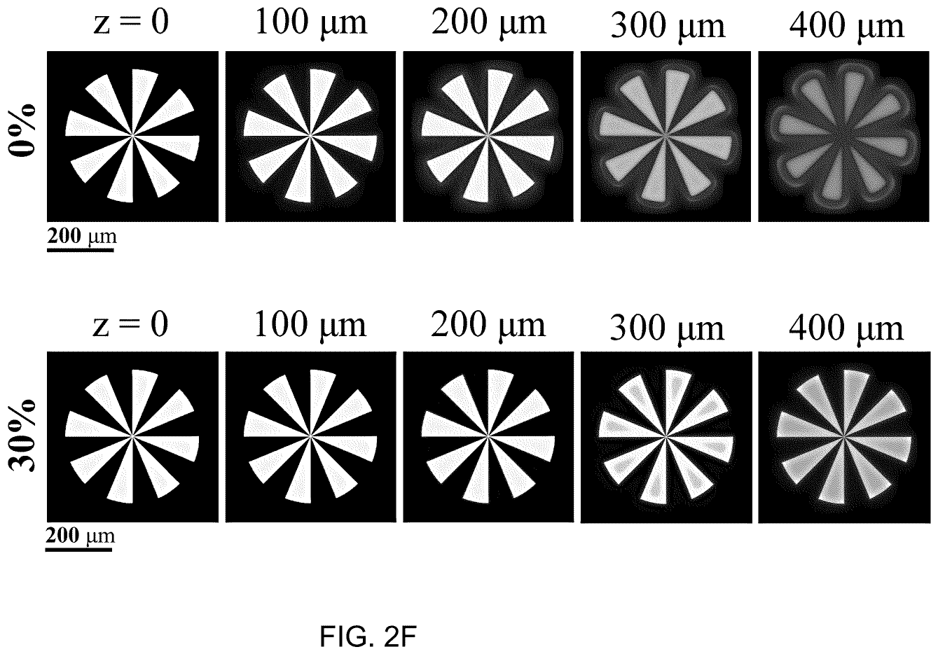

Tests verified that IDX can effectively tune the refractive index by measuring the refractive index of bioinks comprising 5% GelMA and various concentrations of IDX at the working wavelength of the bioprinter (405 nm). For refractive index measurement, mixtures of 5% GelMA with 0%, 20%, 25%, 30%, or 35% IDX, and PBS as solvent were prepared. As shown in FIG. 2A, the refractive index of the bioink linearly increases when increasing the IDX concentration from 20% to 35%. The refractive indices of bioinks comprising 5% GelMA and 0% IDX, as well as the as-purchased IDX solution (OptiPrep™, Sigma-Aldrich) which contains 60% IDX, are also plotted for reference.

The scattering effect of the material is usually characterized by scattering coefficient, anisotropy, and reduced scattering coefficient. To further verify that a proper concentration of IDX can effectively reduce the bioinks' scattering effect, bioinks comprising 5% GelMA, 40 million/mL 293T cells, and various IDX concentrations (0%, 20%, 25%, 30%, or 35%) were prepared. An approach that combined Monte-Carlo simulation and particle swarm optimization algorithm was used to determine the bioink's scattering properties at the working wavelength of the bioprinter (405 nm). The measured scattering coefficient, anisotropy, and reduced scattering coefficient of these bioinks are shown in FIGS. 2B-D, respectively. The high scattering coefficients and the anisotropy values that are close to 1 suggest that the cell-laden bioinks are highly light-scattering, with mostly forward scattering. The bioink without refractive index tuning has a scattering coefficient of 11.76 mm⁻¹, and a reduced scattering coefficient of 0.164 mm⁻¹. By tuning the refractive index with 30% IDX, the scattering coefficient of the bioink significantly decreases to 1.377 mm⁻¹, and the reduced scattering coefficient decreases to 0.014 mm⁻¹, which means this approach can reduce the scattering by approximately 10-fold. By carefully tuning the concentration of contrast agent, scattering can potentially be further reduced.

In order to provide a more intuitive result to visualize how the refractive-index-matched bioink can reduce the scattering effect, a Monte-Carlo approach was also used to simulate the angular distribution and spatial distribution of the scattered light. FIG. 2E shows the simulated angular distribution of travel directions of those photons' passing through a 1 mm thick bioinks containing 40 million/mL cells at two different IDX concentrations. As indicated, the photons widely spread out when traveling in the 0% IDX bioink, while they are still highly aligned in the 30% IDX bioink. Furthermore, using a spoke-like pattern projected onto the bioink, and assuming this pattern is infinitely collimated, the simulation results in FIG. 2F show that this pattern quickly blurs out in the bioink with 0% IDX, while it can mostly preserve its details in the bioink with 30% IDX.

IDX has been generally considered biocompatible and non-toxic. It is iso-osmolar and chemically inert, making it compatible with a wide range of bioinks and cell types.

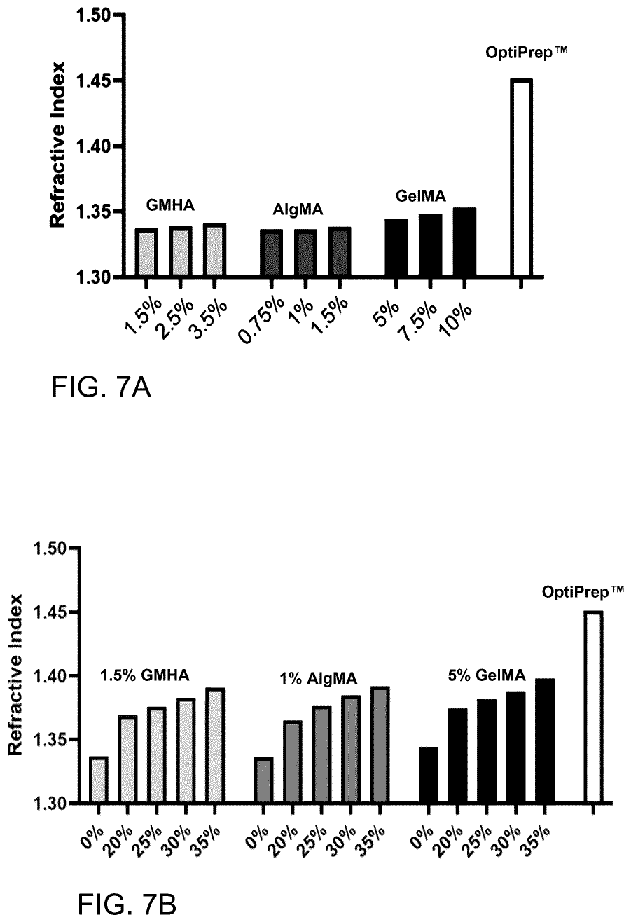

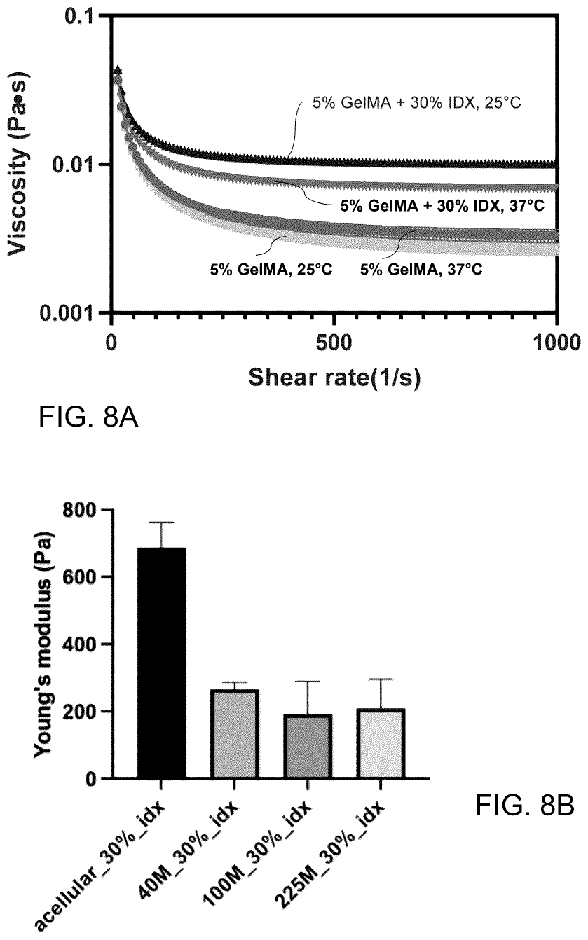

In order to verify IDX's biocompatibility with various biomaterials and cell types, we performed thin slab bioprinting using three commonly used formulas: human umbilical vein endothelial cells (HUVEC) in GelMA, human Schwann cells (HSC) in glycidyl methacrylate hyaluronic acid (GMHA), and C2C12 in alginate methacrylate (AlgMA). FIGS. 7A and 7B are plots of the refractive indices of these common bioinks at various biopolymer and IDX concentrations, respectively. Since the ideal IDX concentration range varies between 20% and 35%, we chose to compare 3D printed tissues in which 0% or 35% IDX was used. As will be apparent to those of skill in the art, other bioinks may be used including, but not limited to, multi-arm PEG (polyethylene glycol), Gel-NB (Gelatin-norbornene), Gel-SH (Gelatin-thiol), and combinations thereof, including HA-NB, HA-SH, etc. Furthermore, the sample cell types described herein are not intended to be limiting but were selected to evaluate the process's impact on viability of mammalian cells. Applications of the inventive approach go well beyond mammalian tissue/organ fabrication. Adaptation for bioprinting using non-mammalian, plant, algal, fungal, bacterial, and archaea cells, and combinations thereof, will be apparent to those in the art based on the disclosure herein.

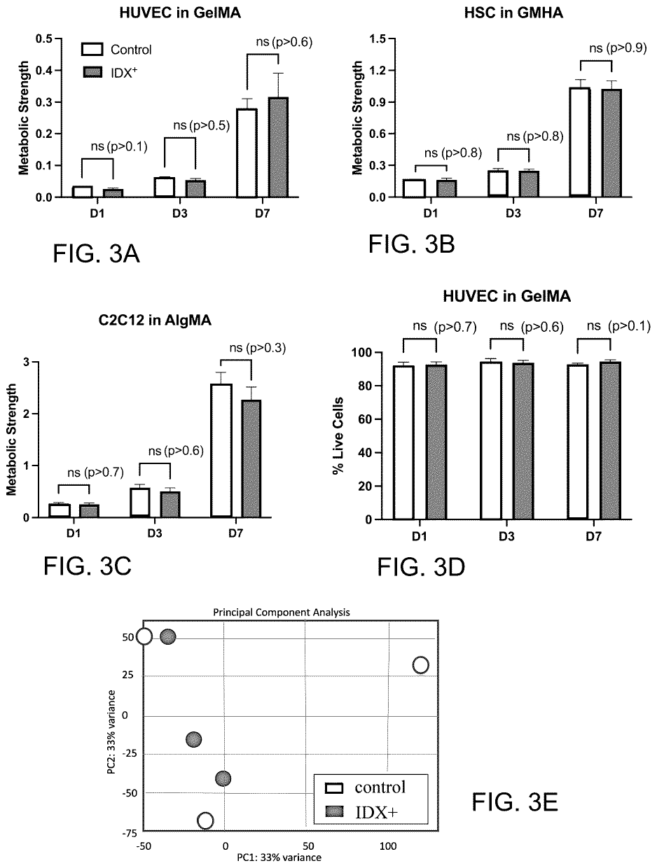

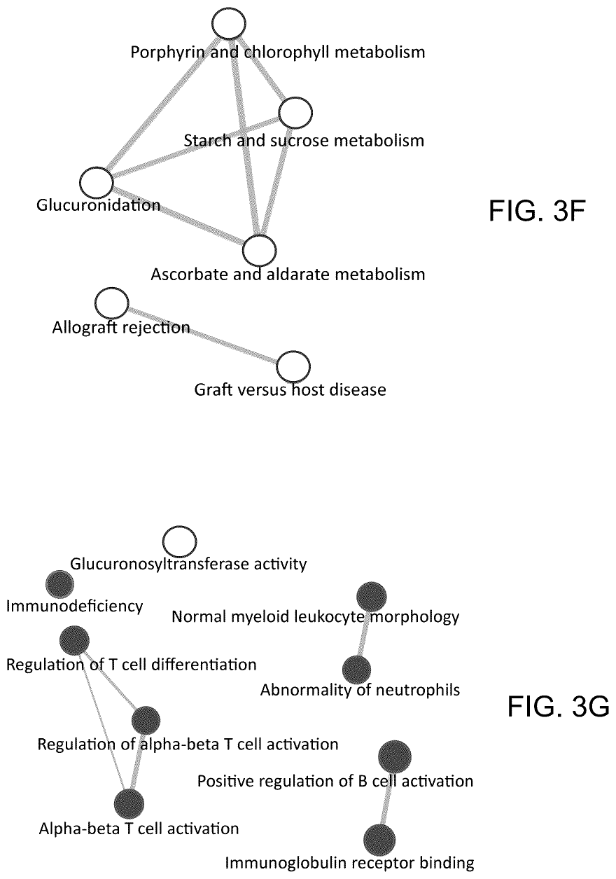

The bioink used in HUVEC viability experiment (FIGS. 3A, 3D) consists of 5% GelMA, 0.6% LAP, 2 million/mL HUVEC cells, 0% or 35% IDX, and PBS as solvent. The bioink used in HSC viability experiment (FIG. 3B) consists of 2.5% GMHA, 0.5% GelMA, 0.6% LAP, 10 million/mL HSC cells, 0% or 35% IDX, and PBS as solvent. The bioink used in C2C12 viability experiment (FIG. 3C) consists of 1% AlgMA, 0.25% GelMA, 0.6% LAP, 10 million/mL C2C12 cells, 0% or 35% IDX, and PBS as solvent. The bioink used in HUVEC RNAseq experiment (FIG. 3G) consists of 5% GelMA, 0.6% LAP, 10 million/mL HUVEC cells, 0% or 35% IDX, and PBS as solvent.

Metabolic activity strength measured using the CCK8 assay reveals exponential growth of the encapsulated cells in all three types of bioinks across the 7 days of culturing. Furthermore, using IDX has no statistically significant effect (n=6, p>0.1 for all pairs) on cells' metabolic activity compared to controls, meaning that IDX did not hinder the proliferation of cells, as seen in FIGS. 3A-C).

In addition, live/dead staining was also used to characterize cell viability. HUVEC in GelMA bioink is printed as thin slabs with either 0% or 35% IDX. Overall, greater than 90% cell viability was observed for Day 1, Day 3, and Day 7 of culture (FIG. 3D). No statistically significant difference (n=4, p>0.1 for all pairs) was observed between 0% and 35% IDX, meaning that the incorporation of IDX was found to not significantly affect cell viability. Furthermore, the cytoskeleton of HUVECs was stained using phalloidin to visualize the cells' morphology. HUVECs in both experimental conditions demonstrated endothelialization at day 7, with no qualitative differences observed.

In addition to confirming that HUVEC viability and angiogenesis was not significantly inhibited in IDX-incorporated bioinks versus that of the controls, we also evaluated whether any phenotypic and metabolic alterations were induced by the presence of IDX. This is especially important for vasculature-on-a-chip studies, where the metabolism and immune-regulation of the endothelium is of interest. Clinically observed adverse effects and toxicological studies have identified elevation of oxidative stress as the main mechanism of IDX-induced endothelial dysfunction, and heme oxygenase-1 is upregulated to counteract the injury. Here, RNA-sequencing (RNA-seq) was performed to comprehensively investigate the potential changes caused by IDX exposure in the HUVECs in 3D-printed GelMA hydrogel.

Differential expression was first investigated with DESEQ2. In the principal component analysis (PCA), the samples did not cluster into two groups (FIG. 3E), indicating that significant phenotype alternation was not induced by IDX. On the other hand, 587 genes have been significantly upregulated and 569 genes downregulated, with a cutoff of |log 2-FoldChange|>0.5849 and p<0.05. To further evaluate the biological impact that IDX brought to the bioprinted vasculature, Gene Set Enrichment Analysis (GSEA) using the Molecular Signature Database (MSigDB) was used. In the hallmark gene sets collection (h.all.v7.4.symbols.gmt [Hallmarks]), xenobiotic metabolism, heme metabolism, and complement gene sets were identified as significantly enriched in the samples printed with bioink containing IDX with FDR<25% and p<0.05). The upregulation of xenobiotic metabolism indicated that biotransformation and the relevant enzyme expression was activated by IDX. The heme metabolism is believed to be a result of the oxidative stress induced by the IDX, and the enrichment in the complement system is potentially related to inflammatory responses. These findings correlate well with the clinically observed adverse effects and previous toxicological studies of IDX. Since the enrichment was observed in samples collected 7 days after bioprinting when IDX should have been fully dissipated from the slabs, it is necessary to take the delayed and long-lasting molecular changes in HUVECs into consideration for further studies.

Within the curated gene sets collection (c2.all.v7.4.symbols.gmt [Curated]), 3 gene sets were identified to be upregulated and 11 gene sets were downregulated in response to IDX exposure; in the ontology gene sets collection (c5.all.v7.4.symbols.gmt [Gene ontology]), 18 gene sets were identified to be upregulated and 1 gene set was downregulated. As highlighted by the network analysis by Cytoscape, porphyrin and chlorophyll metabolism, glucuronidation, starch and sucrose metabolism and ascorbate and aldarate metabolism were downregulated (FIG. 3F), along with the downregulation of glucuronosyltransferase activity (FIG. 3G), which is potentially related to the activation of xenobiotic metabolism. Meanwhile, the allograft reject and graft versus host disease gene sets were downregulated (FIG. 3F), while a group gene sets related to acquired immunity (regulation of T cell differentiation, alpha beta T cell activation & regulation of alpha beta T cell activation; normal myeloid leukocyte morphology & abnormality of neutrophils; immunoglobulin receptor binding & positive regulation of B cell activation) was upregulated accompanied by the upregulation of immunodeficiency (FIG. 3G), implying a immunosuppressive regulatory role of the endothelium induced by IDX.

Collectively, several notable changes in metabolic and immune-regulatory activities of the endothelial cells are observed upon exposure to IDX during bioprinting, which should be taken into consideration in further studies in which pharmacokinetics and pharmacodynamic (PK/PD), immunotherapy evaluation and pathological progression are of the focus. However, the incorporation of IDX in the bioink did not hinder the viability and endothelialization of the HUVECs, and the phenotype of the HUVECs showed minimal change due to exposure to IDX.

Vasculature networks are essential in large-scale engineered tissues or organs. Although 3D printed porous structures such as 3D lattices or log pile geometries can well support the cell viability in thick engineered tissues under an in-vitro culture condition, the absence of vascular networks makes them unable to be integrated with the host vasculature upon transplantation. Thus, biofabrication of pre-vascularized thick tissue has attracted intense research interest. To date, direct 3D fabrication of vasculature networks with high cell density remains a major challenge. Some studies use sacrificial material to cast the vasculature networks, followed by dissolving the sacrificial templates. Since the sacrificial materials contain no or low density of cells, a high fabrication resolution can be achieved. However, the post-fabrication processes involving sacrificial template removal and endothelial cells seeding/settling are complicated and time-consuming (a few hours to a day), and the perfusion culture cannot be started until finishing these post-processing steps. As a result, the cell viability within the thick tissues is compromised. Also, due to the effect of gravity, the seeded endothelial cells will be nonuniformly deposited in the vascular network. Hence, direct 3D printing of pre-vascularized tissues with encapsulated endothelial cells provides a more promising approach, because the encapsulated endothelial cells can migrate to and proliferate at the lumen, enabling endothelialization of the pre-vascularized network and promoting angiogenesis.

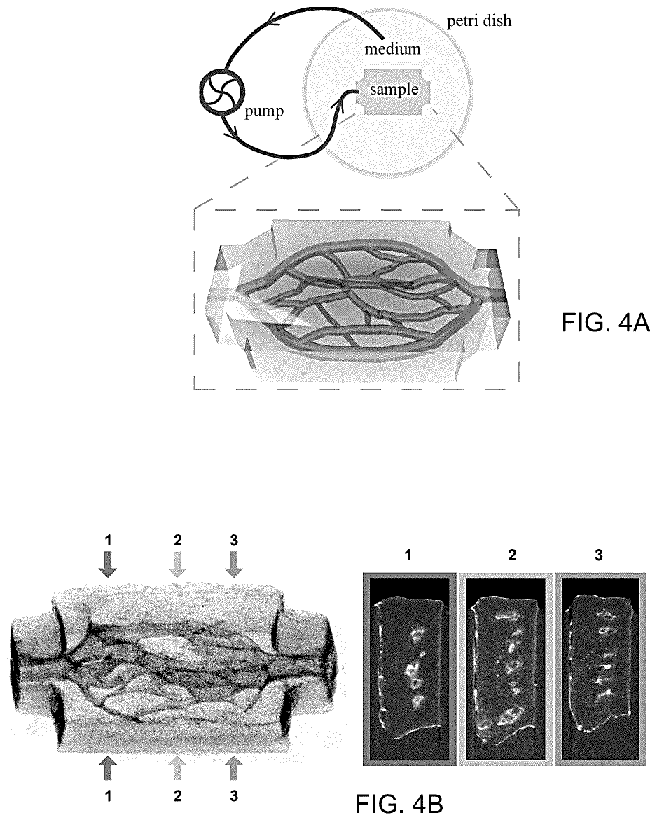

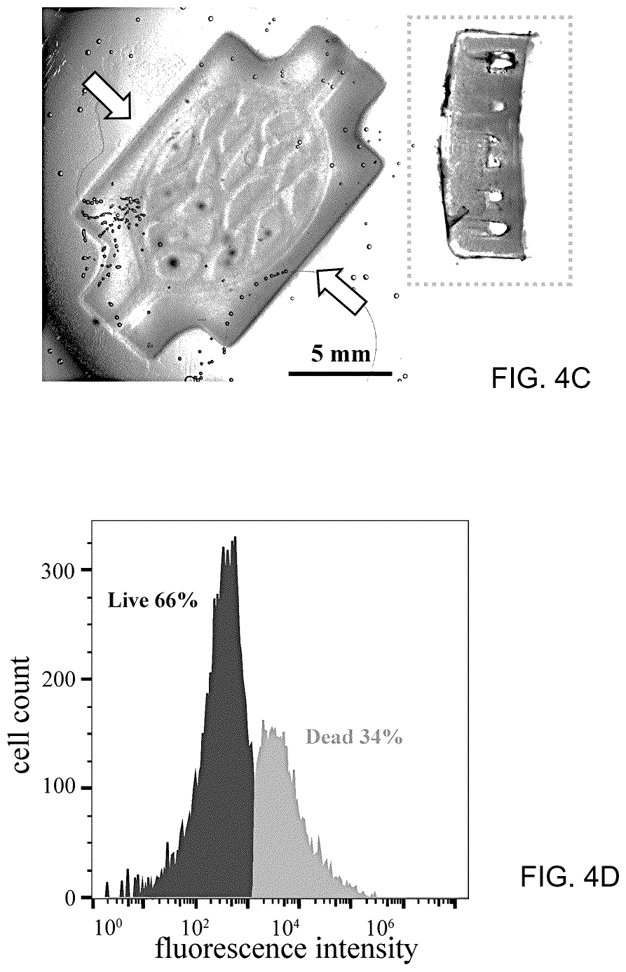

Due to the Density-Viability-Resolution Trilemma, earlier research on direct printing of pre-vascularized tissues is usually limited to either no/low cell density or low fabrication resolution. A large-scale (17×11×3.6 mm) pre-vascularized tissue construct was designed and 3D printed using a refractive-index-matched bioink containing 40 million/mL cells. The bioink consists of 5% GelMA, 0.6% LAP, 1% yellow food dye, 30% IDX, 23 million/mL HUVEC, 17 million/mL HDF, and PBS as solvent. A diagram of the model is shown in the lower portion of FIG. 4A.

GelMA, GMHA, and LAP were synthesized following established protocols. IDX solution was purchased from Sigma-Aldrich (OptiPrep™, D1556-250ML, Sigma-Aldrich).

The diameters of the hollow vascular channels range from 250 μm to 600 μm. HUVECs and human dermal fibroblasts (HDFs) were encapsulated in the GelMA bioink at 23 million/mL and 17 million/mL density, respectively. FIG. 4B shows the micro-CT images (perspective view and cross-sections) of the 3D printed structure, and FIG. 4C shows the bright field microscopic images (top view and cross-section) of the printed structure. Hollow channels were observed in the scaffold, supporting the claim that desired complex microstructural features can be printed in cellularized scaffolds with high resolution and high fidelity. Fluorescence imaging of the printed tissue, where the two cell types were stained with CellTracker Green and CellTracker Orange, respectively, confirmed that high-density, uniformly mixed cells were encapsulated in the printed structures.

Due to the vasculature networks, pre-vascularized 3D tissues have a much larger surface area than non-vascularized 3D tissues, thus the gas and nutrient exchange efficiencies can be greatly improved. Although the diffusion limit can be overcome by adding vasculature networks to the 3D engineered tissues, under static culture conditions, the exchange efficiencies are still insufficient to support the viability of high-cell-density, large-scale tissues. Therefore, it is necessary to use perfusion culture with the pre-vascularized tissues. Gravity-driven perfusion is widely used for perfusion culture, however, for large-scale tissues with complex vasculature networks, the pressure provided by gravity is insufficient to drive the culture medium through such high-resistance vasculature networks. A perfusion culture driven by a peristaltic pump is an alternative solution that provides increased control over the system and scalability. As shown in FIG. 4A, a microfluidic peristaltic pump was used to actively pump the culture medium through the vasculature network immediately after 3D bioprinting. The system was maintained for 14 days in an incubator, and then the printed construct was harvested. Flow cytometry was used to count the number of live/dead cells (stained with Zombie Green viability kit) in the harvested tissue. FIG. 4D plots the results, showing that 66% of live cells were highly viable in this thick tissue across the 14 days perfusion culture, suggesting that necrosis was avoided due to the 3D printed vasculature and perfusion culture.

The harvested samples were subjected to immunofluorescence staining to evaluate changes in cell morphology and function in response to perfusion culture. DAPI and phalloidin were used to stain the nucleus and cytoskeleton, and VE-cadherin was used to label the cellular junctions between HUVECs. Immunofluorescence imaging of cryosection in a plane perpendicular to the channels revealed vascular channels merging and/or splitting. A monolayer of HUVECs was observed around the channel's cross-section, confirming the endothelialization of the 3D printed vascular channels. In addition, immunofluorescence imaging from various horizontal planes (z slices) further confirmed that a dense and uniform monolayer of endothelial cells was formed along the printed lumen. More importantly, angiogenesis beyond the printed lumen was observed in the maximum projection images stacking all z layers, indicating potential sites of spontaneous formation of new capillaries. Immunofluorescence imaging confirmed that the cells in large 3D printed vascular networks are viable, functional, and replicate key morphological changes that are observed in vascular networks in vivo.

The following non-limiting examples provide additional details relating to the materials and methods used in evaluation of embodiments of the inventive approach as well as further applications and refinements of the approach.

### Example 1: Determining the Optimal Concentration of Iodixanol

Cytoplasm typically has a refractive index between 1.36 and 1.39. Assuming the IDX linearly changes the refractive index of the bioink, the concentration of IDX should be in the range between 20% and 35%. However, the optimal concentration is difficult to determine before 3D printing because it depends on many factors including the bioink composition, cell type, osmolarity, temperature, light wavelength, etc.

One approach for determining the optimal concentration is to make bioinks with a series of IDX concentrations and without photoinitiator, and then use an optical goniometer type setup to measure the scattered light distribution of a 1 mm thick bioink.

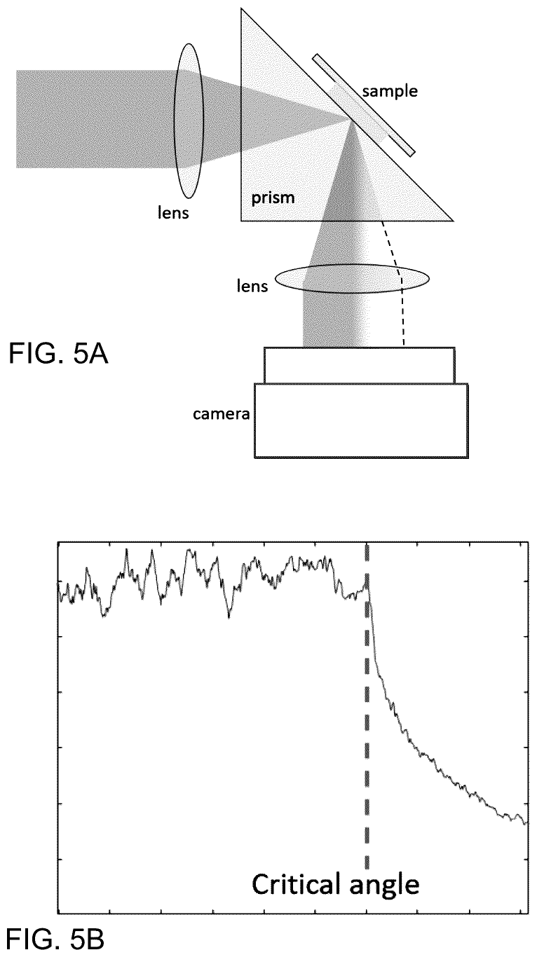

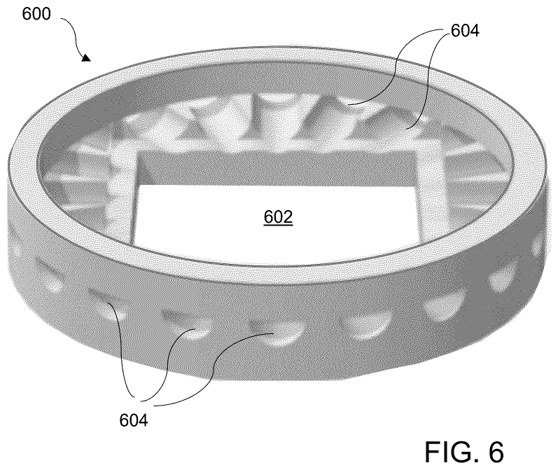

The refractive index of bioink can be measured using a simple refractometer with a 405 nm laser. As shown in FIG. 5A, the laser beam is focused on the hypotenuse of a right-angle prism, which is made of N-SF11 glass and has a refractive index of 1.8421 at 405 nm. The converging beam enters the prism-sample interface at different angles, and total internal reflection (TIR) happens at some of these angles. The diverging beam exits the prism, is collimated by a lens, and collected by a camera. The pattern collected by the camera shows the boundary where TIR happens, and the critical angle of TIR can be determined based on this pattern (FIG. 5B). In order to accurately relate the TIR boundary position on the captured image to the actual reflecting angle, water and isopropanol, whose refractive index and thus TIR critical angles are known, were used to calibrate this relation.

Generally, it can be challenging to directly measure the scattering coefficient and anisotropy in a highly scattering specimen such as a biological tissue or high-cell-density bioink. Indirect methods involving numerical simulation are usually required to accurately analyze these properties.

We used a well-developed 3D Monte-Carlo method combined with a particle swarm optimization algorithm (57) to numerically calculate the scattering properties of the cell-laden bioinks. The total transmittance and total reflectance at 405 nm of the 1 mm thick bioink containing 40 million/mL cells were measured using a UV-Vis-NIR spectroscope (Perkin Elmer, Lambda 1050) with integrating sphere. The angular intensity distribution of the scattered light at 405 nm was measured with a simple optical goniometer type setup. Given arbitrary optical properties, the total transmittance, total reflectance, and angular distribution can be calculated by the Monte-Carlo method. We then used a particle swarm optimization algorithm on MATLAB (MathWorks, Natick MA) to acquire the optical properties which best fit the measured total transmittance, total reflectance, and angular distribution. FIG. 2E provides sample line graphs showing that the simulated angular distribution of the scattered light well fits the measurement values. Next, we used the acquired scattering coefficient and anisotropy to simulate the spatial distribution of light inside the bioink with Monte-Carlo method. This spatial distribution is also the point spread function of scattering. Hence, the pattern scattering results shown in FIG. 2F are the convolution of this point spread function and the original pattern.

### Example 2: Cell Culture

Primary HUVEC and primary HDF were purchased from Cell Applications, Inc. (San Diego, CA) and cultured per the manufacturer's protocol. Briefly, HUVECs are maintained in the endothelial growth medium (211-500, Cell Applications, Inc.), fed with fresh medium every 2 days and passaged 1:5 every 4 days. HDFs are maintained in the fibroblast growth medium (116-500, Cell Applications, Inc.) and passage 1:3 every 3 days. Cells under passage 7 were used for bioprinting and further studies.

HSC, C2C12 and 293T were purchased from ATCC (Manassas, VA) and cultured in Dulbecco's Modified Eagle Medium (DMEM, 11995-065, Gibco) supplemented with 10% (v/v) fetal bovine serum (FBS, 10438026, Gibco). The cells were passaged every 3 days.

Prior to printing, the cells are washed with Dulbecco's phosphate-buffered saline (DPBS, 14190144, Gibco) and disassociated with 0.25% Trypsin-EDTA (25200072, Gibco) for 3 minutes in a 37° C. CO₂ incubator, followed by neutralization with their culture media and resuspension in the bioink.

3D printed thin slabs were rinsed with PBS three times to remove IDX residue, and then cultured in an incubator, fed with fresh medium every 2 days.

### Example 3: Tissue Perfusion Culture and Evaluation

3D printed large scale tissues were briefly rinsed, and then immediately connected to the perfusion culture system. Referring to FIG. 4A, the perfusion culture system used a peristaltic pump to continuously feed fresh medium through the vascular channels at a flow rate of 0.2 ml/min. 30 mL of endothelial growth medium was added into the reservoir (petri-dish). Medium was changed 3 times in the first hour of perfusion culture to remove residual IDX and was changed every 2 days during the subsequence culture period. The tissues were harvested at Day 14.

The perfusion culture system was constructed as follows:

Using a FDM 3D printer (Prusa MK3S+, Prusa Research), an open-top fluidic manifold 600 was constructed out of polyethylene terephthalate glycol (PETG). The square hole 602 in the center accommodates the glass coverslip substrate upon which the DLP 3D printed tissue scaffold is adhered to. The open channels 604 radiating away from the center provide multiple degrees of freedom through which perfusion tubing can be threaded to interface with the scaffold's inlet/outlet. The manifold's total diameter is small enough to be placed inside a standard 90 mm petri dish, in which 30 mL of media was placed as a reservoir.

Using a SLA 3D printer (Form 3+, Formlabs) and open source protocols (58), we constructed a small scale multi-channel microfluidic peristaltic pump. The central component of the system comprises a NEMA 17 stepper motor. The motor is connected to a 3D printed central shaft mounted on bearings to allow free spinning; the central shaft itself possesses six stainless steel rollers, also mounted on bearings to allow free spinning. This assembly is then enclosed within a grooved manifold through which peristaltic tubing can be threaded. Programmable control of the motor's operation then rotates the central shaft and associated rollers against the peristaltic tubing, resulting in the repeated roller-induced contraction/expansion cycles on the tubing that produce peristaltic flow.

Immediately after DLP 3D bioprinting, the iodixanol-modified, HUVEC-laden tissue scaffold adhered to the glass coverslip was placed within the open-top fluidic manifold 600. Peristaltic tubing (silicone, ID 1 mm, McMaster-Carr) threaded through the pump was connected to the tissue scaffold's inlet via Tygon tubing (ID 0.5 mm, Cole-Parmer) and secured using tissue adhesive (3M VetBond).

HUVECs were 3D printed in 250 μm slabs using GelMA bioink with 0% or 35% IDX. After 7 days of culture, the RNAs from the 3D printed slabs are extracted with TRIzol reagent (15596018, Ambion) followed by purification using spin column method with Direct-zol RNA Microprep (R2060, Zymo Research). The RNA quality evaluation and sequencing was performed by Novogen Inc. The sequencing data was analyzed with FastQC, trimmed with Trimmomatic, aligned with HISAT2, annotated with StringTie. Differential gene expression was analyzed with DESeq2. Gene set enrichment analysis was performed with GSEA_4.2.3 (Broad Institute). Network analysis was performed with Cytoscape 3.9.1 using the EnrichmentMap application.

Cell viability in the printed thin slabs was quantified with Cell Counting Kit-8 (CCK-8, K1018, ApexBio). At designated time points, the slabs are washed with DPBS, incubated with 1mL fresh media with 10% CCK-8 reagent at their regular incubation condition for 45 minutes. After incubation, 200 μL supernatant was collected from each sample and their OD at 450nm was measured with Tecan Infinite 200 pro.

The cell viability was also evaluated with Live/Dead staining. At designated time points, the slabs were washed with DPBS twice, incubated with 2 μM Calcein AM (C3099, Invitrogen) and 3 μM propidium iodide (P3566, Invitrogen) in the fresh culture media at their regular incubation condition for 30 min. After incubation, the slabs were washed with DPBS twice and imaged with Leica DMI6000B fluorescence microscope. The live cell percentage was counted using particle analysis tool in ImageJ.

Cell viability in the thick tissue was characterized by Zombie Green viability kit (#423111, BioLegend). The tissue was cultured in a perfusion culture system for 14 days, and then harvested. A thin section was taken from the middle portion of the sample using a scalpel. The section was stained with Zombie Green viability kit, next fixed with 4% PFA, and then digested using 0.25% trypsin-EDTA to remove the GelMA. Cells were strained using a 40 μm filter before loading onto the BD Accuri C6 Plus Flow Cytometer. 15,000 cells were collected for analysis. Cells were first gated to exclude the cell debris. The remaining cells were gated based on the fluorescent intensity in the FL1 channel (488 nm).

The soft hydrogel samples need to be scanned in an aqueous environment to avoid deformation of the samples. However, the radiology contrast between the GelMA hydrogel and water is very low. Therefore, a non-water-soluble contrast agent is needed. In this study, we chose BaCO₃ as the contrast agent. The sample was first soaked in 1% w/V BaCl₂ solution, and then transferred to 1% w/V Na₂CO₃ solution, immediately followed by Na₂CO₃ perfusion. Thus, the external surface of the construct and the internal surface of the vascular network were coated with BaCO₃.

Samples were scanned using a Skyscan 1076 μCT scanner (Bruker, Konich, Belgium) immersed in PBS in a custom-designed 3D printed container. Samples were scanned at 9 μm×9 μm×9 μm voxel size, applying an electrical potential of 50 kVp, a current of 200 μA, 180° in 0.8 ° steps, and using a 0.5 mm Al filter. All μCT image processing was performed using MATLAB. Volumetric data was reconstructed and viewed using the Volume Viewer Application.

The large-scale tissues were processed into cryosections or chunks followed by immunofluorescence staining and imaging.

The harvested samples were fixed with paraformaldehyde (PFA) for 30 min, and then soaked overnight in 30% sucrose solution at 4° C. on a nutating tube rocker. Next, they were immersed in optimal cutting temperature (O.C.T.) compound (23-730-571, Fisher Scientific), and placed in a cryostat set at −20° C. Cryosections of 40 μm or 60 μm thick were made and placed on poly-l-lysine (0.1% w/v) coated slides.

The cryosectioned samples were gently washed with DPBS, permeabilized with 0.1% Triton-X-100(T 8787 , Sigma) and blocked with 2% bovine serum albumin (A2153, Sigma). Primary rabbit VE-Cadherin antibody (2158, Cell Signaling Technology) was diluted 1:200 in cell staining buffer (420201, BioLegend) and incubated with the samples at 4° C. overnight. The primary antibody was then labeled with donkey anti-rabbit IgG CF543 secondary antibody (20308-1, Biotium), which was diluted in cell staining buffer at 1:200 and incubated at 37° C. for 2 hours. Cytoskeleton and nuclei were labeled with Phalloidin eFluor 660 (50-6559-05, eBioscience) and DAPI (4083S, Cell Signaling Technology) per the manufacturer's instruction before the slides were mounted with antifade reagent (9071S, Cell Signaling Technology).

The harvested samples were fixed with paraformaldehyde (PFA) for 30 min, and vertically cut into 4 pieces using a scalpel. Next, the small pieces were horizontally split into two halves to obtain chunks that expose half of the printed vascular channels. These chunks were cleared using a tissue clearing kit (CytoVista, V11322, ThermoFisher) to facilitate imaging per the manufacturer's protocol. The primary and secondary antibody was diluted 1:200 in the antibody dilution buffer, respectively. Phalloidin 660 and DAPI were diluted 1:1000 and co-incubated with the secondary antibody.

In the cryosection process, the samples slightly swelled (~20%) due to the sucrose treatment. In the chunk process, the sample significantly shrunk (~2× to 3×) due to the tissue clearing. FIG. 4C represents the actual size of the as-printed samples.

The stained samples were imaged on Leica SP8 fluorescence confocal microscope, Leica DMI6000B fluorescence microscope, and Keyence BZX800 fluorescence microscope.

### Example 4: Stiffness Prediction

The mechanical properties of the extracellular matrix (ECM) are known to impact cell phenotype and behavior. The curing of hydrogels that can be used as ECM mimics occurs through free-radical polymerization, where factors such as light power, exposure time, and the concentrations of both the precursor solution and the photoinitiator significantly affect the stiffness of the resulting material.

As disclosed herein, contrast agents can be incorporated into the hydrogel precursor solution to tune the refractive index closer to that of the encapsulated cells' cytoplasm to minimize the scattering effect. Different cells exhibit variance in refractive index due to their organelle composition. Consequently, the hydrogel precursor composition is also varied to match each cell type's optimal contrast agent concentration. Although it is possible to measure the stiffness for each cell type under comprehensive printing conditions, a significant challenge arises when dealing with precious cells, such as primary cells isolated from tissues.

A neural network (NN) machine learning model was employed to predict the construct stiffness in HCD printing based on cell density and light exposure time. Initially, stiffness results under detailed printing conditions are obtained using a low-cost 293T cell line. After training the model with data from 293T cells, transfer learning can be implemented to predict the stiffness of a model cell, HepG2 cells, using only a few data points from HepG2 under limited printing conditions. This model demonstrates a good fitting with the stiffnesses from 293T cells and effectively predicts the stiffness of HepG2 cells with limited input data.

To obtain stiffness data from HCD printing with 293T cells, we encapsulated cells at various densities in 5% GelMA hydrogel, subjecting them to different light exposure conditions. Compression tests were applied to determine the stiffness of the prints. It was observed that at a constant cell density, an increase in exposure time resulted in a stiffer sample, while at a consistent exposure time, higher cell density led to a softer sample. These results align with expectations: prolonged light exposure generates more free radicals to polymerize the precursor solution, forming a denser network, whereas higher cell density increases light scattering, thus reducing polymerization efficiency. Utilizing a 3-layer neural network (NN) model, a 3D plot was generated to illustrate the distribution of stiffness under conditions where cell density ranges from 0 to 200 million, and exposure time varies from 0 to 40 seconds.

The quality of the fully trained model was evaluated using mean absolute error (MAE) and coefficient of determination (R2) metrics. The MAE metric calculates the difference between the model predicted stiffness and the actual measured stiffness, then takes the average of the absolute errors. Greater MAE error value implies more prediction error. The MAE metric has the benefit of interpretability by giving the average pascal error on model predictions. The R2 metric is another commonly used method to evaluate the prediction quality of statistical models. The R2 value tells us the proportion of the explained variance of the data by the prediction model. Greater R2 value implies better fitting of the model with the maximum of 1, which means a perfect fit.

A typical machine learning evaluation process would separate a portion of the data away from the model training process and apply the evaluation metrics on that standalone evaluation data set. However, unlike typical big data-based machine learning, only a limited amount of data was available in the field of bioprinting due to high cost and long experiment processing time. Therefore, every data point is precious and contributes to the quality of the resulting NN model. To properly evaluate the NN model, both MAE and R2 metrics were first applied to the model trained with all available 293T cell-based data, comparing the predictions to the training data set. A low MAE error and a high R2 value were observed, demonstrating that the fully trained NN model nicely fit the training data. However, it was uncertain whether this model could precisely predict the stiffness with previously unseen printing conditions. Leave-one-out (LOO) cross validation, a special case of k-fold cross validation, was applied. The k-fold cross validation separates the available data set into a predetermined number of k folds of subsets. At every iteration, this method trains the model on the data set by leaving out one of the subsets, then evaluates the trained model on the left-out subset. After repeating for all k folds, the averaged evaluation score would be a reasonable quality assessment for the model on this particular data set. Note that on each iteration of the k-fold cross validation, the left-out subset of data used to evaluate the model is unseen by this model during the training process. Therefore, the k-fold cross validation result also accounts for the generalizability of the model to unseen data in the given data distribution. Based on that, the LOO cross validation is simply a special case of k-fold cross validation where k equals the total number of data points, which means only one data point is left out each time. The LOO cross validation is known to be unbiased and is suitable for small data problems.

LOO cross validation of the NN model on the 293T cell data revealed that testing error on the unseen left-out data was mostly under 500 Pa with the exception of four conditions corresponding to edge cases with the longest exposure time within the experiment range. The result indicates that the NN model trained on the given data set is likely to have less than 500 Pa error on predicting the unseen data within the parameter ranges, i.e., having from 0 to 200 M/mL 293T cell density and having exposure time within 40 s, while having chances to give faulty predictions on the edge case with 40 s exposure time.

Next, HepG2 cells were selected as the model for applying transfer learning due to their larger cell size with different scattering properties and their expression of distinct biomarkers in response to the stiffness of ECM. In order to utilize the previously trained NN model based on 293T cell data, transfer learning was applied to train the new model that fits to the HepG2 data. Due to the pretrained base model, the new model was able to quickly adapt to the new HepG2 cell type with a limited amount of training data available, namely nine different printing conditions. After evaluating with LOO cross validation, similar trends were observed: the model was able to predict stiffness in the given range with less than 500 Pa error with the exception of edge conditions. Reasonable stiffness predictions were achieved with the NN model trained using strictly limited amounts of data. The model will be able to achieve better prediction quality as increased amounts of data are collected. These results also demonstrate that the edge cases are the most critical data points for training a successful model which could guide the future data collection process. The NN prediction method is easily adaptable to new cell types with even less data by applying transfer learning, leading to precision bioprinting with scalable stiffness control at low cost.

### Example 5: Cell Viability in Hdc Bioprinted Hepatocytes

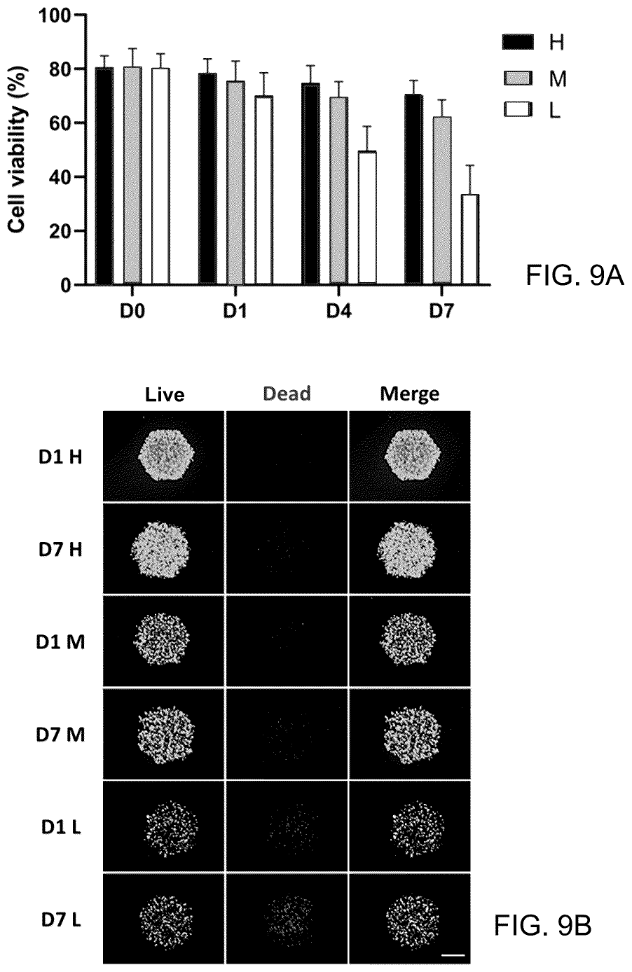

Bioinks, which consist of individual cells and hydrogels, usually have cell densities ranging from 0.1 to 10 million cells per cm³, at least one order of magnitude lower than the physiological levels found in native tissues like the liver. By incorporating refractive index matching into the HCD printing method via the use of a contrast agent, tests demonstrated that HCD bioprinting of mouse primary hepatocytes (MPHs) can achieve good viability. This can be attributed to closer cell-cell interactions and a greater extent of cell reorganization observed in the HCD bioprinting model. Hepatocyte densities of 80 M/ml (High), 20 M/ml (Medium) and 5 M/ml (Low) were combined in a bioink mixture of GelMA 6% with 30% IDX. FIG. 9A plots of cell viability at differential hepatocyte density over the evaluation period of 1 week at days 0, 1, 4 and 7. FIG. 9B provides fluorescent images of live/dead staining of differential hepatocyte density after one week culture.

Over the course of 7 days, MPHs formed aggregates with each other in the HCD (80 million) group, demonstrating the capacity to sustain viability over an extended duration when compared to hepatocytes cultured in a low cell density format.

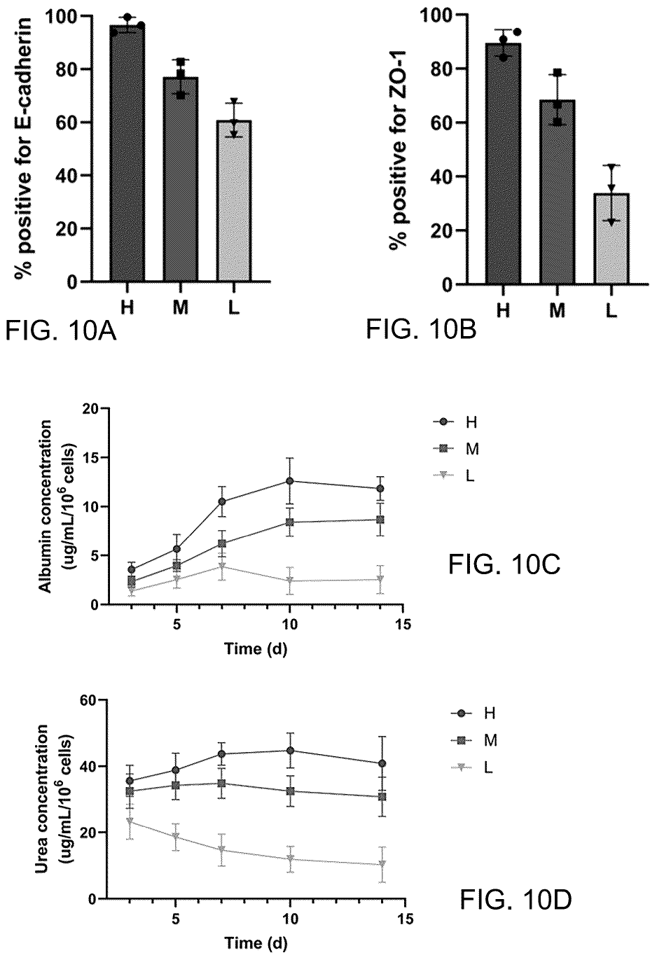

To further study the HCD aggregates, analyses on E-cadherin, an epithelial marker known to protect primary hepatocytes from apoptosis, ZO1, a tight junction marker, and intracellular albumin, a functional hepatic marker, were performed. Immunofluorescent staining and flow cytometry demonstrated enhanced albumin secretion, cell-cell interactions (E-cadherin), and cell-cell tight junctions in the HCD group. FIGS. 10A and 10B plot the quantitative analysis by flow cytometry of E-cadherin and ZO 1, respectively. Furthermore, results showed that the anabolic and catabolic functions of the MPHs were maintained to a greater extent with HCD printing, as evidenced by albumin (FIG. 10C) and urea production (FIG. 10D), likely attributed to closer cell-cell contact.

In addition to applications for drug screening, the HCD model may further contribute to improving an understanding of biology by more accurately replicating the genetic profiles of liver tissues. Since we observed that HCD and MCD can significantly enhance metabolic function due to cell-cell interaction, we further compared these two groups using RNA-sequence and qPCR.

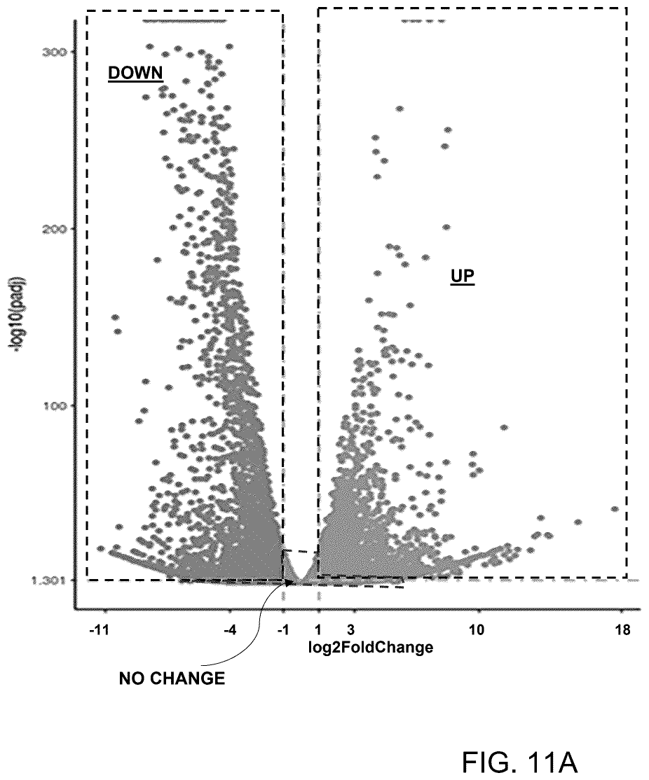

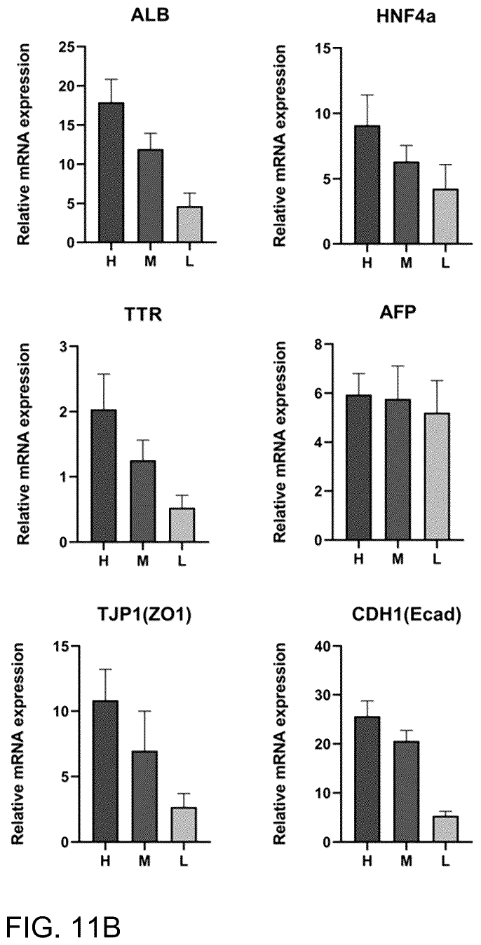

The results revealed a certain degree of differences between the two conditions, with a Spearman rank correlation coefficient of 0.76. The volcano plot in FIG. 11A indicated that 7363 DEGs (|log 2(fold change)|>1, Q value<0.05) were labeled on the map, providing evidence of distinct gene expression between HCD and MCD printing. Relative expression levels of hepatic marker genes we compared in the HCD and MCD groups. Referring to FIG. 11B, the expression levels of hepatic markers highly expressed in mature hepatocytes, such as HNF4a, TTR, and ALB, were higher in the HCD condition. The expression levels of the fetal hepatic marker AFP were not significantly different between the two conditions. Additionally, the upregulation of TJP1 (ZO1) and CDH1 (Ecad) in HCD further supports the notion that HCD printing can enhance cell-cell interaction.

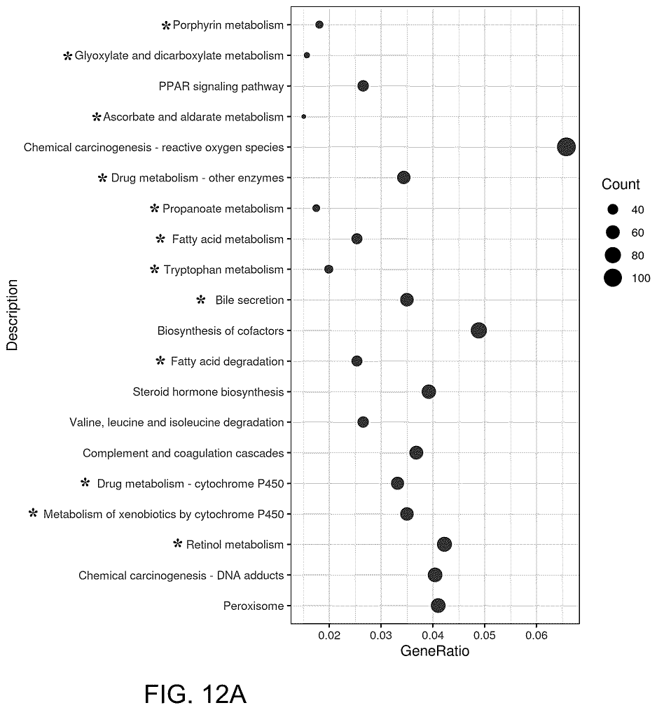

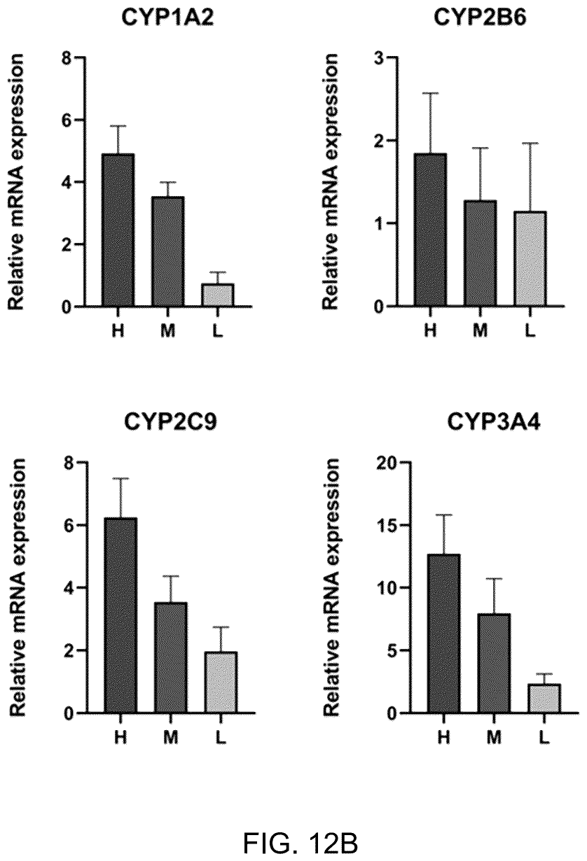

We further performed KEGG pathway/GO enrichment analyses between HCD and MCD models based on RNA-seq. As shown in FIG. 12A, the differentially expressed genes (DEGs) were enriched in 20 major signaling pathways, including liver metabolic pathways such as retinol metabolism, drug metabolism-cytochrome P450, and metabolism of xenobiotics by cytochrome P450. The starred descriptions to the left of the chart represent liver related pathways.

Metabolic activity of hepatocytes is central to their function and largely depends on the activity of enzymes in the cytochrome P450 family. We measured the gene expression of cytochromes 1A2, 2B6, 2C9, and 3A4 and found that all were upregulated in the HCD compared to the MCD condition FIG. 12B plots gene expression levels measured for CYP family (CYP1A2, CYP2B6, CYP2C9 and CYP3A4) for High, Medium, and Low cell densities.

Additional challenges arise from the fact that high cell density can reduce adhesive area and alter cell shape, leading to the inactivation of RhoA and subsequent reduction of stress fibers in the actin cytoskeleton. This inactivation can occur through both Hippo kinases-dependent and-independent mechanisms, resulting in the inactivation of YAP/TAZ. Adherens junctions (AJs) protein E-cadherin and tight junctions (TJs) protein ZO1, in confluent cells, trans-dimerize and subsequently stimulate the MST1/2-LATS1/2 kinase cascade to inhibit the activities of YAP/TAZ.

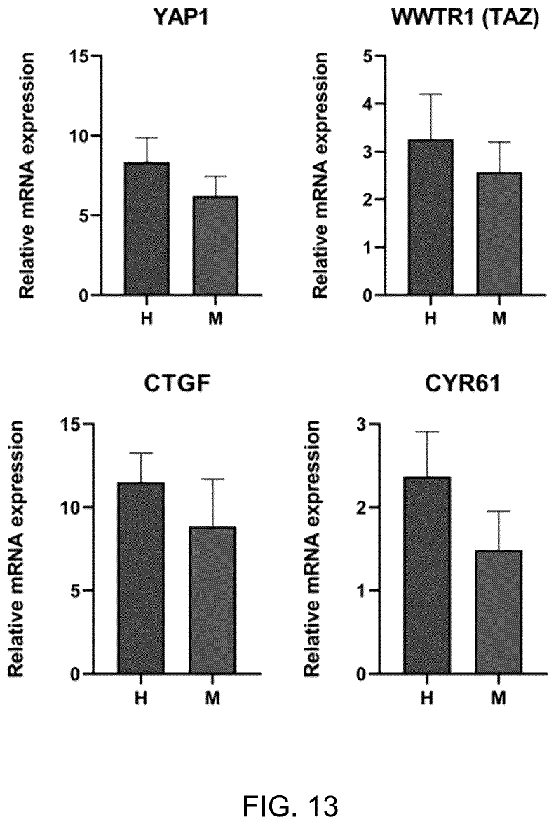

It was hypothesized that the improved hepatocyte maturation and metabolic function in HCD printing could be due to decreased expression of YAP/TAZ, as there is evidence that tight and adherens junction expression can be enhanced by HCD printing through stronger cell-cell interaction. RT-PCR revealed that the expression of YAP1 (YAP), WWTR1 (TAZ), and their target genes CTGF and CYR61 were all lower in HCD (“H”) compared to MCD (“M”), as shown in FIG. 13.

### Example 6: Evaluation of Different Contrast Agents

With the primary evaluation being conducted using IDX as the contrast agent for tuning the bioink's refractive index, additional testing was conducted to confirm that other known water-soluble contrast agents would provide comparable results for purposes of the inventive approach. Specifically, testing was performed using ioversol, sold commercially under the name “Optiray,” and iohexol, sold as “Omnipaque”. Both contrast agents are FDA approved for use in medical imaging.

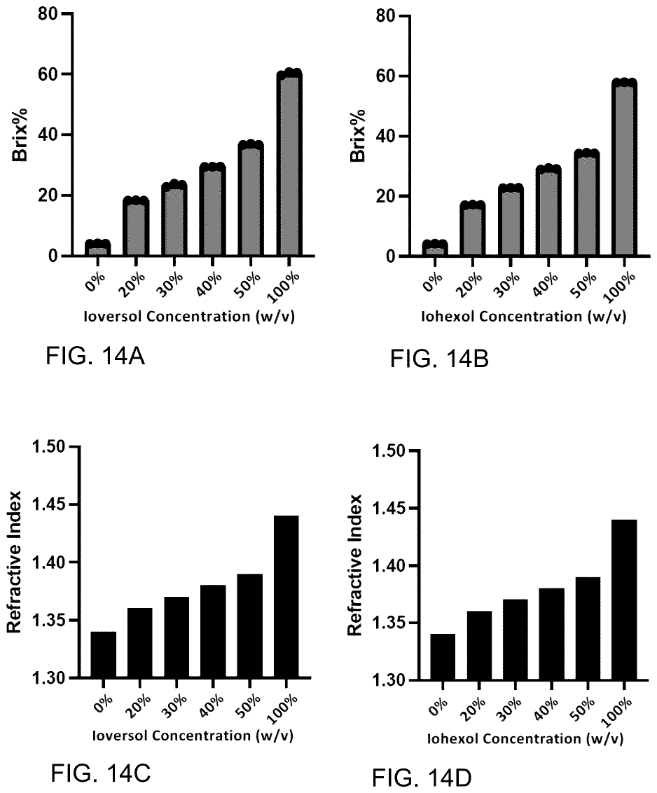

FIGS. 14A and 14B plot the results of measurement using a Brix refractometer of 5% GelMA bioink with various concentrations (0, 20, 30, 40, 50% w/v) of the contrast agents ioversol and iohexol, respectively. For comparison, a 100% (w/v) sample of each contrast agent was also measured. Since this method measures the number of dissolved solids in a liquid based on its specific gravity, the units are in Brix %. FIGS. 14C and 14D show the Brix % data converted to refractive index. These results confirm that additional contrast agents are compatible with the bioink and can effectively tune its refractive index.

By tuning the refractive index of the bioinks through the use of a higher refractive index additive such as a contrast agent, e.g., IDX, in DLP-based 3D bioprinting, high-cell-density and better fabrication resolution can be achieved. The light scattering effect can be significantly reduced by ~10 fold, and the fabrication resolution can be substantially improved. The inventive approach enables 3D bioprinting with high cell density, high viability, and high resolution simultaneously. This technique is straightforward and generalizable and can be easily applied to most biomaterials and cell types, facilitating the fabrication of functional bioartificial tissue for use as models in preclinical and clinical testing and, eventually. artificial organs for implantation.

1. A method for improved resolution of 3D bioprinted tissue comprising:
adding to a photopolymerizable bioink composition having cells encapsulated therein a contrast agent, wherein the bioink composition has a first refractive index and the contrast agent has a second, higher refractive and is added in an amount sufficient to cause the composition to substantially match a refractive index of the encapsulated cells.

2. The method of claim 2, wherein the contrast agent is biocompatible water-soluble contrast agent.

3. The method of claim 1, wherein the contrast agent is selected from iodixanol, iohexol, iopamidol, iopromide, ioversol, iopromide and ioversol.

4. The method of claim 1, wherein the contrast agent has a concentration within a range of 1 to 100% (w/V).

5. The method of claim 1, wherein the contrast agent is iodixanol and has a concentration within a range of 20 to 35 % (w/V).

6. The method of claim 1, wherein the encapsulated cells are selected from cell types consisting of mammalian, non-mammalian, plant, algae, fungal, bacterial, and archaea.

7. The method of claim 1, wherein the encapsulated cells have a cell density in a range of 5 million/mL to 5 billion/mL.

8. The method of claim 7, wherein the cell density is configured to produce a selected tissue stiffness.

9. The method of claim 1, wherein the bioink composition is selected from methacrylated gelatin (GelMA), glycidyl methacrylate hyaluronic acid (GMHA), alginate methacrylate (AlgMA), multi-arm polyethylene glycol (multi-arm PEG), gelatin-norbornene (Gel-NB), Gelatin-thiol (Gel-SH), and combinations thereof.

10. The method of claim 1, wherein the bioink composition is 0.05% to 50% (w/V) GelMA in a solvent.

11. A composition for 3D bioprinting of tissue comprising a mixture of:
a photopolymerizable bioink having a first refractive index;
cells comprising cytoplasm having a second refractive index higher than the first refractive index; and
a contrast agent having a third refractive index higher than the first refractive index, wherein the contrast agent is added in a sufficient amount to substantially match a refractive index of the composition to the second refractive index.

12. The composition of claim 11, wherein the contrast agent is biocompatible water-soluble contrast agent.

13. The composition of claim 11, wherein the contrast agent is selected from iodixanol, iohexol, iopamidol, iopromide, ioversol, iopromide and ioversol.

14. The composition of claim 11, wherein the contrast agent has a concentration within a range of about 1 to 100% w/V.

15. The composition of claim 11, wherein the contrast agent is iodixanol and has a concentration within a range of 20 to 35% w/V.

16. The composition of claim 11, wherein the cells are selected from cell types consisting of mammalian, non-mammalian, plant, algae, fungal, bacterial, and archaea.

17. The composition of claim 16, wherein the encapsulated cells have a cell density in a range of 5 million/mL to 5 billion/mL.

18. The composition of claim 17, wherein the cell density is configured to produce a selected tissue stiffness.

19. The composition of claim 11, wherein the bioink composition is selected from methacrylated gelatin (GelMA), glycidyl methacrylate hyaluronic acid (GMHA), alginate methacrylate (AlgMA), polyethylene glycol (PEG), gelatin-norbornene (Gel-NB), Gelatin-thiol (Gel-SH), and combinations thereof.

20. The composition of claim 11, wherein the bioink composition is 0.05% to 50% (w/V) GelMA in a solvent.
